(19) United States
(12) Patent Application Publication
(10) Pub. No.: US 2009/0088457 A1
(43) Pub. Date: Apr. 2, 2009

(54) Primary Amines as Renin Inhibitors

(75) Inventors: Olivier Bezencon, Riehen (CH); Daniel Bur, Therwil (CH); Olivier Corminboeuf, Allschwil (CH); Corinna Grisostomi, Allschwil (CH); Lubos Remen, Allschwil (CH); Sylvia Richard-Bildstein, Dietwiller (FR); Thomas Weller, Binningen (CH)

Correspondence Address: HOXIE & ASSOCIATES LLC, 75 MAIN STREET , SUITE 301, MILLBURN, NJ, 07041, US

(73) Assignee: Actelion Pharmaceuticals, Ltd., Allschwil (CH)

(21) Appl. No.: 12/224,590

(22) PCT Filed: Mar. 1, 2007
(86) PCT No.: PCT/IB2007/050671

§ 371 Date: Aug. 29, 2008

(30) Foreign Application Priority Data
Mar. 3, 2006 (IB) PCT/IB06/50678

(51) Int. Cl. A61K 31/426; C07D 277/20; C07D 417/02; A61K 31/427; A61P 9/00; A61P 25/00; A61P 13/12; A61K 31/454; C07D 401/14; C07D 213/02

(52) U.S. Cl. 514/342; 548/146; 546/256; 546/329; 514/365

(57) ABSTRACT

The invention relates to novel primary amine derivatives and the use thereof as active ingredients in the preparation of pharmaceutical compositions. The invention also concerns related aspects including processes for the preparation of the compounds, pharmaceutical compositions containing one or more of those compounds and especially their use as inhibitors of renin.

The present invention was made as a result of activities undertaken within the scope of a research collaboration agreement between Merck & Co., Inc., Actelion Pharmaceuticals Ltd, and Actelion Ltd. The agreement was executed on Dec. 4, 2003.

The invention relates to novel compounds of the formula (I). The invention also concerns related aspects including processes for the preparation of the compounds, pharmaceutical compositions containing one or more compounds of formula (I) and especially their use as renin inhibitors in cardiovascular events and renal insufficiency.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). ACE inhibitors do not inhibit Chymase. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, renin inhibitors with good oral bioavailability and long duration of action are required. Recently, the first non-peptide renin inhibitors were described which show high in vitro activity (Oeffier C. et al., Chem. Biol., 1999, 6, 127; Patent Application WO 97/09311; Marki H. P. et al., Il Farmaco, 2001, 56, 21). However, the development status of these compounds is not known.

The present invention relates to renin inhibitors of a non-peptidic nature and of low molecular weight. Described are orally active renin inhibitors of formula (I) which have a long duration of action and which are active in indications beyond blood pressure regulation where the tissular renin-chymase system may be activated leading to pathophysiologically altered local functions such as renal, cardiac and vascular remodelling, atherosclerosis, and possibly restenosis. So, the present invention describes these non-peptidic renin inhibitors of formula (I).

In particular, the present invention relates to novel compounds of the formula (I)

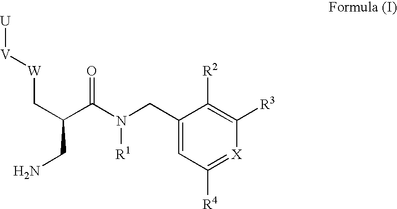

wherein
X represents CH, N, or N⁺—O⁻;

W represents a para-substituted pyridinyl or a thiazolyl, such as especially:

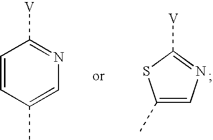

V represents —CH₂CH₂CH₂—, —CH₂CH₂-A-, —CH₂-A-CH₂—, -A-CH₂CH₂—, —CH₂CH₂CH₂CH₂—, -A-CH₂CH₂CH₂—, —CH₂-A-CH₂CH₂—, —CH₂CH₂-A-CH₂—, —CH₂CH₂CH₂-A-, -A-CH₂CH₂—B— (preferred), —CH₂CH₂CH₂CH₂CH₂—, -A-CH₂CH₂CH₂CH₂—, —CH₂-A-CH₂CH₂CH₂—, —CH₂CH₂-A-CH₂CH₂—, —CH₂CH₂CH₂-A-CH₂—, —CH₂CH₂CH₂CH₂-A-, -A-CH₂CH₂CH₂—B—, —CH₂-A-CH₂CH₂—B—, -A-CH₂CH₂—B—CH₂—, -A-CH₂CH₂CH₂—B—CH₂—, —CH₂-A-CH₂CH₂CH₂—B—, —O—CH₂-Q-, wherein Q is bound to the group U of formula (I), or preferably a pyrrolidinyl of the formula:

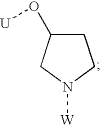

U represents unsubstituted aryl, especially phenyl; mono-, di-, tri- or tetra-substituted aryl (especially mono- di-, tri-, or tetra-substituted phenyl), wherein the substituents are independently selected from the group consisting of C₁₋₇-alkyl (such as especially methyl), —CF₃, halogen, and hydroxy-C₁₋₇-alkyl (such as especially CH₃CH(OH)—); or a five-membered heteroaryl with two heteroatoms independently selected from nitrogen, oxygen and sulphur (preferably pyrazolyl or isoxazolyl), wherein said heteroaryl radical is optionally mono-, di- or tri-substituted, wherein the substitutents are independently selected from the group consisting of C₁₋₇-alkyl, C₁₋₇-alkoxy, —CF₃, —OCF₃, and halogen;
Q represents a five-membered heteroaryl with two or three heteroatoms independently selected from O and N;
A and B represent independently from each others —O— or —S—, especially —O—;
R¹ represents C₁₋₇-alkyl or cycloalkyl, preferably cycloalkyl such as especially cyclopropyl;
R² represents halogen or C₁₋₇-alkyl, preferably chloro or methyl;
R³ represents hydrogen, halogen (such as especially chloro), C₁₋₇-alkyl (such as especially methyl), C₁₋₇-alkoxy, or —CF₃; and
R⁴ represents hydrogen; C₁₋₇-alkyl-O—(CH₂)₀₋₄—CH₂—, such as especially CH₃—O—(CH₂)_{1,2}—CH₂—; CF₃—O—(CH₂)₀₋₄—CH₂—; R′₂N—(CH₂)₀₋₄—CH₂—, wherein R′ is independently selected from the group consisting of hydrogen, C₁₋₇-alkyl (optionally but preferably substituted by one to three fluorine), cyclopropyl (optionally substituted by one to three fluorine), cyclopropyl-C₁₋₇-alkyl (optionally but preferably substituted by one to three fluorine), and —C(═O)—R″ wherein R″ is C₁₋₄-alkyl, C₁₋₄-alkoxy, —CF₃, —CH₂—CF₃, or cyclopropyl; or R⁵—C(═O)—(O)₀₋₁—(CH₂)₀₋₄—, wherein R⁵ is C₁₋₄-alkyl, C₁₋₄-alkoxy, or cyclopropyl; wherein R′ and R″ preferably do not both simultaneously represent hydrogen; and salts thereof.

The general terms used hereinbefore and hereinafter preferably have, within this disclosure, the following meanings, unless otherwise indicated:

Where the plural form is used for compounds, salts, pharmaceutical compositions, diseases and the like, this is intended to mean also a single compound, salt, or the like.

Any reference to a compound of formula (I) is to be understood as referring also to salts (especially pharmaceutically acceptable salts) of a compound of formula (I), as appropriate and expedient.

The term C₁₋₇-alkyl, alone or in combination with other groups, means saturated, straight or branched chain groups with one to seven carbon atoms, preferably one to four carbon atoms, i.e. C₁₋₄-alkyl. Examples of C₁₋₇-alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred, especially the methyl and ethyl groups.

The term C₁₋₇-alkoxy, alone or in combination with other groups, refers to an R—O— group, wherein R is a C₁₋₇-alkyl group. Examples of C₁₋₇-alkoxy groups are methoxy, ethoxy, propoxy, iso-propoxy, iso-butoxy, sec-butoxy and tert-butoxy.

The term hydroxy-C₁₋₇-alkyl, alone or in combination with other groups, refers to an HO—R— group, wherein R is a C₁₋₇-alkyl group. Examples of hydroxy-C₁₋₇-alkyl groups are HO—CH₂—, HO—CH₂CH₂—, HO—CH₂CH₂CH₂— and CH₃CH(OH)—.

The term halogen means fluorine, chlorine, bromine or iodine, preferably fluorine, chlorine or bromine. In a more preferred embodiment of the invention the term halogen means fluorine or chlorine.

The term cycloalkyl, alone or in combination with other groups, means a saturated cyclic hydrocarbon ring system with 3 to 7 carbon atoms, i.e. cyclopropyl, cyclobutyl, cyclopentyl, cyclohexyl or cycloheptyl, preferably cyclopropyl.

The term aryl, alone or in combination, refers to a phenyl, naphthyl or indanyl group, preferably a phenyl group.

The expression pharmaceutically acceptable salts encompasses either salts with inorganic acids or organic acids like hydrochloric acid, hydrobromic acid, hydroiodic acid, sulfuric acid, sulfamic acid, phosphoric acid, nitric acid, phosphorous acid, nitrous acid, citric acid, formic acid, acetic acid, oxalic acid, maleic acid, lactic acid, tartaric acid, fumaric acid, benzoic acid, mandelic acid, cinnamic acid, palmoic acid, stearic acid, glutamic acid, aspartic acid, methanesulfonic acid, ethanesulfonic acid, ethanedisulfonic acid, p-toluenesulfonic acid, salicylic acid, succinic acid, trifluoroacetic acid, and the like that are non toxic to living organisms or in case the compound of formula (I) is acidic in nature with an inorganic base like an alkali or earth alkali base, e.g. sodium hydroxide, potassium hydroxide, calcium hydroxide and the like. For other examples of pharmaceutically acceptable salts, reference can be made to “Salt selection for basic drugs”, Int. J. Pharm. (1986), 33, 201-217.

The compounds of the formula (I) may contain asymmetric carbon atoms. Substituents at a double bond or a ring may be present in cis- (=Z-) or trans (=E-) form unless indicated otherwise. The compounds of formula (I) may thus be present as mixtures of stereoisomers or preferably as pure stereoisomers. Mixtures of stereoisomers may be separated in a manner known per se, e.g. by column chromatography, thin layer chromatography, HPLC or crystallization.

Compounds of the invention also include nitrosated compounds of formula (I) that have been nitrosated through one or more sites such as oxygen (hydroxyl condensation), sulfur (sulfhydryl condensation) and/or nitrogen. The nitrosated compounds of the present invention can be prepared using conventional methods known to one skilled in the art. For example, known methods for nitrosating compounds are described in U.S. Pat. Nos. 5,380,758, 5,703,073, 5,994,294, 6,242,432 and 6,218,417; WO 98/19672; and Oae et al., Org. Prep. Proc. Int., 15(3): 165-198 (1983).

A preferred embodiment of the present invention relates to a compound of formula (I), wherein

X represents CH or N; and
R⁴ represents hydrogen; C₁₋₇-alkyl-O—(CH₂)₀₋₄—CH₂—; CF₃—O—(CH₂)₀₋₄—CH₂—; or R′₂N—(CH₂)₀₋₄—CH₂—, wherein R′ is independently selected from the group consisting of hydrogen, C₁₋₇-alkyl (optionally substituted by one to three fluorine), cyclopropyl (optionally substituted by one to three fluorine), cyclopropyl-C₁₋₇-alkyl (optionally substituted by one to three fluorine), and —C(═O)—R″ wherein R″ is C₁₋₄-alkyl, —CF₃, —CH₂—CF₃, or cyclopropyl.

A preferred embodiment of the present invention relates to a compound of formula (I), wherein X represents CH or N⁺—O⁻.

A preferred embodiment of the present invention relates to a compound of formula (I), wherein A and B both represent —O—.

A preferred embodiment of the present invention relates to a compound of formula (I), wherein R¹ represents cyclopropyl.

A preferred embodiment of the present invention relates to a compound of formula (I), wherein W represents

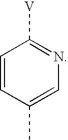

A preferred embodiment of the present invention relates to a compound of formula (I), wherein V represents —O—CH₂CH₂—O—, —CH₂—CH₂—O— wherein the —CH₂ part of —CH₂—CH₂—O— is bound to the group W of formula (I), —O—CH₂-Q-, or

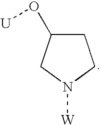

A preferred embodiment of the present invention relates to a compound of formula (I), wherein V represents —O—CH₂CH₂—O— or —O—CH₂-Q-.

A preferred embodiment of the present invention relates to a compound of formula (I), wherein V—W represents:

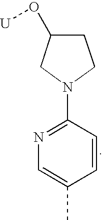

A preferred embodiment of the present invention relates to a compound of formula (I), wherein U represents

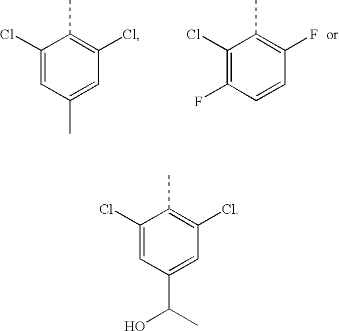

A preferred embodiment of the present invention relates to a compound of formula (I), wherein U represents

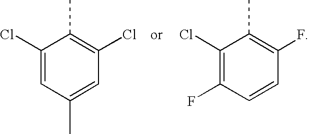

A preferred embodiment of the present invention relates to a compound of formula (I), wherein Q represents an isoxazolyl or an oxadiazolyl.

A preferred embodiment of the present invention relates to a compound of formula (I), wherein Q represents an isoxazolyl, especially an isoxazolyl that is connected to the rest of the molecule of formula (I) as follows:

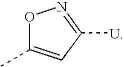

A preferred embodiment of the present invention relates to a compound of formula (I), wherein R² represents Cl, and R³ represents hydrogen.

A preferred embodiment of the present invention relates to a compound of formula (I), wherein R⁴ represents CH₃—O—(CH₂)₂₋₃— or CH₃—C(═O)—NH—CH₂—CH₂—.

A preferred embodiment of the present invention relates to a compound of formula (I), wherein R⁴ represents —CH₂CH₂CH₂—O—CH₃ or —CH₂CH₂—O—CH₃.

A preferred embodiment of the present invention relates to a compound of formula (I), wherein R⁴ represents —CH₂CH₂—O—CH₃.

A preferred embodiment of the present invention relates to a compound of formula (I), wherein the moiety

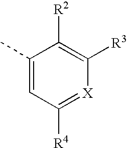

represents one of the following possibilities:

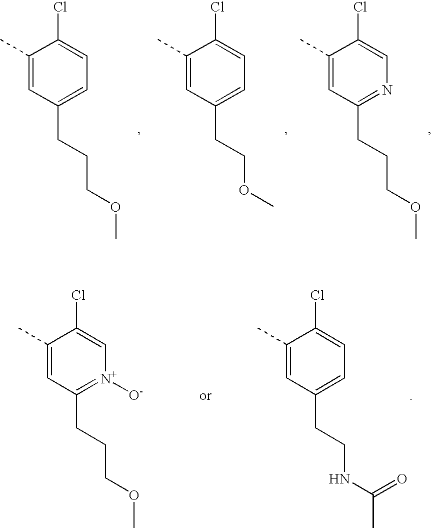

In an especially preferred embodiment, the present invention relates to a compound of formula (I), wherein

X represents CH or N;
W represents a para-substituted pyridinyl or a thiazolyl;
V represents —O—CH₂CH₂—O— or a pyrrolidinyl of the formula:

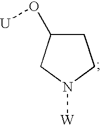

U represents di-, tri-, or tetra-substituted phenyl, wherein the substituents are independently selected from the group consisting of C₁₋₇-alkyl, halogen and hydroxy-C₁₋₇-alkyl;
R¹ represents cyclopropyl;
R² represents halogen or C₁₋₇-alkyl;
R³ represents hydrogen, halogen, or C₁₋₇-alkyl; and
R⁴ represents hydrogen or C₁₋₇-alkyl-O—(CH₂)₀₋₄—CH₂—.

The present invention also relates to compounds of formula (I) wherein the meanings of one or more of the substituents and symbols as defined for formula (I), or a preferred embodiment of formula (I), are replaced by their preferred meanings as defined herein, such as those defined for the above-given preferred embodiments.

A very preferred embodiment of the present invention relates to a compound of formula (I) selected from the group consisting of:
(R)-3-amino-N-cyclopropyl-2-{2-[2-(2,6-dichloro-4-methyl-phenoxy)-ethoxy]-thiazol-5-ylmethyl}-N-(2,3-dimethyl-benzyl)-propionamide,
(R)-3-amino-2-{2-[2-(2-chloro-3,6-difluoro-phenoxy)-ethoxy]-thiazol-5-ylmethyl}-N-cyclopropyl-N-(2,3-dimethyl-benzyl)-propionamide,
(R)-3-amino-N-cyclopropyl-2-{2-[2-(2,6-dichloro-phenoxy)-ethoxy]-thiazol-5-ylmethyl}-N-(2,3-dimethyl-benzyl)-propionamide,
(R)-3-amino-N-cyclopropyl-2-{2-[2-(2,6-dichloro-3,4-dimethyl-phenoxy)-ethoxy]-thiazol-5-ylmethyl}-N-(2,3-dimethyl-benzyl)-propionamide,
(R)-3-amino-2-{2-[2-(2-chloro-6-fluoro-3-methyl-phenoxy)-ethoxy]-thiazol-5-ylmethyl}-N-cyclopropyl-N-(2,3-dimethyl-benzyl)-propionamide,
(R)-3-amino-N-cyclopropyl-2-(2-{2-[2,6-dichloro-4-(1-hydroxy-ethyl)-phenoxy]-ethoxy}-thiazol-5-ylmethyl)-N-(2,3-dimethyl-benzyl)-propionamide,
(R)-3-amino-2-{2-[2-(3-chloro-2,6-difluoro-phenoxy)-ethoxy]-thiazol-5-ylmethyl}-N-cyclopropyl-N-(2,3-dimethyl-benzyl)-propionamide,
(R)-3-amino-N-cyclopropyl-2-{2-[2-(2,6-dichloro-4-fluoro-phenoxy)-ethoxy]-thiazol-5-ylmethyl}-N-(2,3-dimethyl-benzyl)-propionamide,
(R)-2-aminomethyl-N-cyclopropyl-N-(2,3-dichloro-benzyl)-3-{2-[(R)-3-(2,6-dichloro-4-methyl-phenoxy)-pyrrolidin-1-yl]-thiazol-5-yl}-propionamide,
(R)-2-aminomethyl-N-cyclopropyl-N-(2,3-dichloro-benzyl)-3-{2-[(R)-3-(2,6-dichloro-phenoxy)-pyrrolidin-1-yl]-thiazol-5-yl}-propionamide,
(R)-2-aminomethyl-N-cyclopropyl-N-(2,3-dichloro-benzyl)-3-{2-[(R)-3-(2,6-dichloro-3,4-dimethyl-phenoxy)-pyrrolidin-1-yl]-thiazol-5-yl}-propionamide,
(R)-2-aminomethyl-3-{2-[(R)-3-(2-chloro-6-fluoro-3-methyl-phenoxy)-pyrrolidin-1-yl]-thiazol-5-yl}-N-cyclopropyl-N-(2,3-dichloro-benzyl)-propionamide,
(R)-2-aminomethyl-N-cyclopropyl-N-(2,3-dichloro-benzyl)-3-(2-{(R)-3-[(R)-2,6-dichloro-4-(1-hydroxy-ethyl)-phenoxy]-pyrrolidin-1-yl}-thiazol-5-yl)-propionamide,
(R)-2-aminomethyl-N-cyclopropyl-N-(2,3-dichloro-benzyl)-3-(2-{(R)-3-[(S)-2,6-dichloro-4-(1-hydroxy-ethyl)-phenoxy]-pyrrolidin-1-yl}-thiazol-5-yl)-propionamide,
(R)-2-aminomethyl-3-{2-[(R)-3-(3-chloro-2,6-difluoro-phenoxy)-pyrrolidin-1-yl]-thiazol-5-yl}-N-cyclopropyl-N-(2,3-dichloro-benzyl)-propionamide,
(R)-2-aminomethyl-N-cyclopropyl-N-(2,3-dichloro-benzyl)-3-{2-[(R)-3-(2,6-dichloro-4-fluoro-phenoxy)-pyrrolidin-1-yl]-thiazol-5-yl}-propionamide,
(R)-2-aminomethyl-N-[2-chloro-5-(3-methoxy-propyl)-benzyl]-N-cyclopropyl-3-{6-[(R)-3-(2,6-dichloro-4-methyl-phenoxy)-pyrrolidin-1-yl]-pyridin-3-yl}-propionamide, and
(R)-2-aminomethyl-N-[5-chloro-2-(3-methoxy-propyl)-pyridin-4-ylmethyl]-N-cyclopropyl-3-{6-[(R)-3-(2,6-dichloro-4-methyl-phenoxy)-pyrrolidin-1-yl]-pyridin-3-yl}-propionamide.

A further very preferred embodiment of the present invention relates to a compound of formula (I) selected from the group consisting of:
(R)-2-aminomethyl-N-[2-chloro-5-(2-methoxy-ethyl)-benzyl]-N-cyclopropyl-3-{6-[2-(2,6-dichloro-4-methyl-phenoxy)-ethoxy]-pyridin-3-yl}-propionamide,
(R)-2-aminomethyl-N-[2-chloro-5-(3-methoxy-propyl)-benzyl]-N-cyclopropyl-3-{6-[2-(2,6-dichloro-4-methyl-phenoxy)-ethoxy]-pyridin-3-yl}-propionamide,
(R)-2-aminomethyl-N-[2-chloro-5-(2-methoxy-ethyl)-benzyl]-N-cyclopropyl-3-{6-[(R)-3-(2,6-dichloro-4-methyl-phenoxy)-pyrrolidin-1-yl]-pyridin-3-yl}-propionamide,
(R)-2-aminomethyl-N-[5-chloro-2-(3-methoxy-propyl)-pyridin-4-ylmethyl]-N-cyclopropyl-3-{6-[2-(2,6-dichloro-4-methyl-phenoxy)-ethoxy]-pyridin-3-yl}-propionamide, and
(R)-2-aminomethyl-N-[2-chloro-5-(2-methoxy-ethyl)-benzyl]-N-cyclopropyl-3-{6-[(S)-3-(2,6-dichloro-4-methyl-phenoxy)-pyrrolidin-1-yl]-pyridin-3-yl}-propionamide.

The compounds of formula (I) are useful for the treatment and/or prophylaxis of diseases such as or related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases related to the renin-angiotensin system.

The compounds of formula (I) are especially useful for the treatment and/or prophylaxis of hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In one embodiment, the invention relates to a method for the treatment and/or prophylaxis of diseases, which are associated with a dysregulation of the renin-angiotensin system, in particular to a method for the treatment and/or prophylaxis of the above-mentioned diseases, said methods comprising administering to a patient a pharmaceutically active amount of a compound of formula (I).

A further aspect of the present invention relates to pharmaceutical compositions comprising a compound of formula (I) and a pharmaceutically acceptable carrier material. These pharmaceutical compositions may be used for the treatment and/or prophylaxis of the above-mentioned diseases. The pharmaceutical compositions can be used for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The invention also relates to the use of a compound of formula (I) for the preparation of pharmaceutical compositions for the treatment and/or prophylaxis of the above-mentioned diseases.

The production of the pharmaceutical compositions can be effected in a manner which will be familiar to any person skilled in the art (see for example Mark Gibson, Editor, Pharmaceutical Preformulation and Formulation, 1HS Health Group, Englewood, Colo., USA, 2001; Remington, The Science and Practice of Pharmacy, 20th Edition, Philadelphia College of Pharmacy and Science) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Compounds of formula (I) or the above-mentioned pharmaceutical compositions are also of use in combination with other pharmacologically active compounds such as ACE-inhibitors, neutral endopeptidase inhibitors, aldosterone antagonists, angiotensin II receptor antagonists, endothelin receptors antagonists, vasodilators, calcium antagonists, potassium activators, diuretics, sympatholitics, beta-adrenergic antagonists, alpha-adrenergic antagonists, 11beta-hydroxysteroid dehydrogenase type 1 inhibitors, soluble guanylate cyclase activators and/or other drugs beneficial for the prevention or the treatment of the above-mentioned diseases.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The compounds of formula (I) can be manufactured by the methods outlined below, by the methods described in the examples or by analogous methods.

An aryl bromide or heteroaryl bromide of type A, wherein U, V and W are as defined for formula (I), can be converted into the corresponding aldehyde of type B, as described in Scheme 1. A Knoevenhagel condensation yields a compound of type C. Reduction of the double bond yields a compound of type D. An amide coupling yields a compound of type E, and final reduction of the nitrite leads a compound of formula (I).

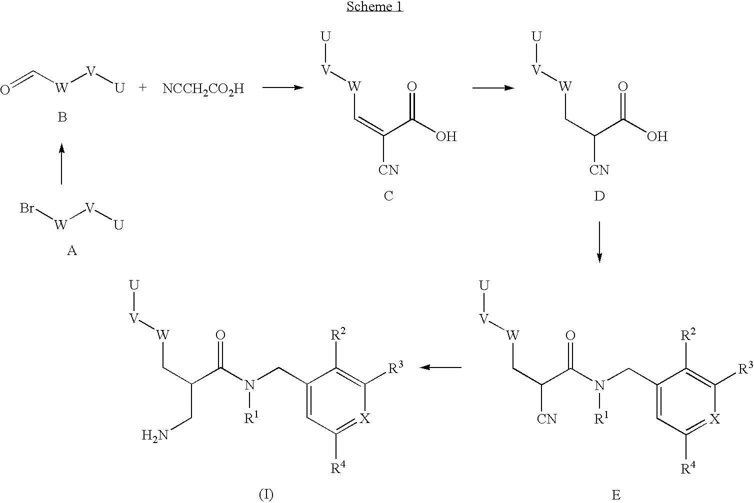

Sometimes the U—V—W—Br fragment cannot be prepared as such, or is not suitable for the subsequent chemistry. In this case, a fragment V^{a}—W—Br of type F, as described in Scheme 2, can be prepared, wherein V^{a} stands for a precursor of the V^{a} substituent. The V^{a}-substituent can then be modified along the synthesis. The same chemistry as described in Scheme 1 leads to the compounds of types G, H, J, and K, respectively. Completion of the U—V—W-fragment leads to a compound of type E.

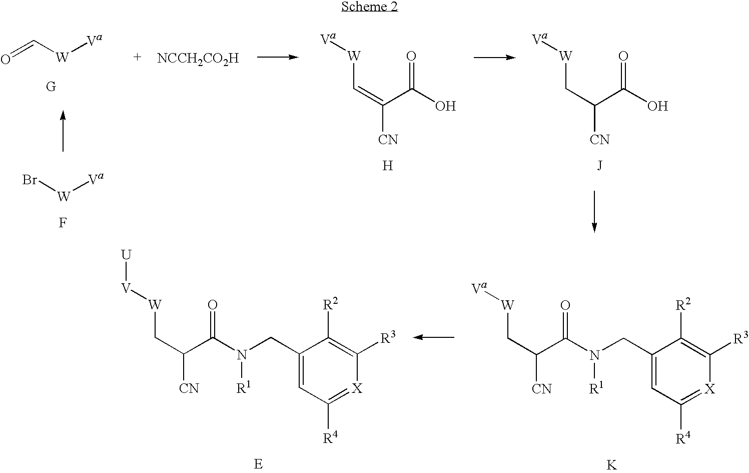

Also, a compound of type K can be reduced into a compound of type L, as represented in Scheme 3. Protection with a protecting group PG leads to a compound of type M. Completion of the U—V—W fragment leads to a compound of type N. Final deprotection leads to a compound of formula (I).

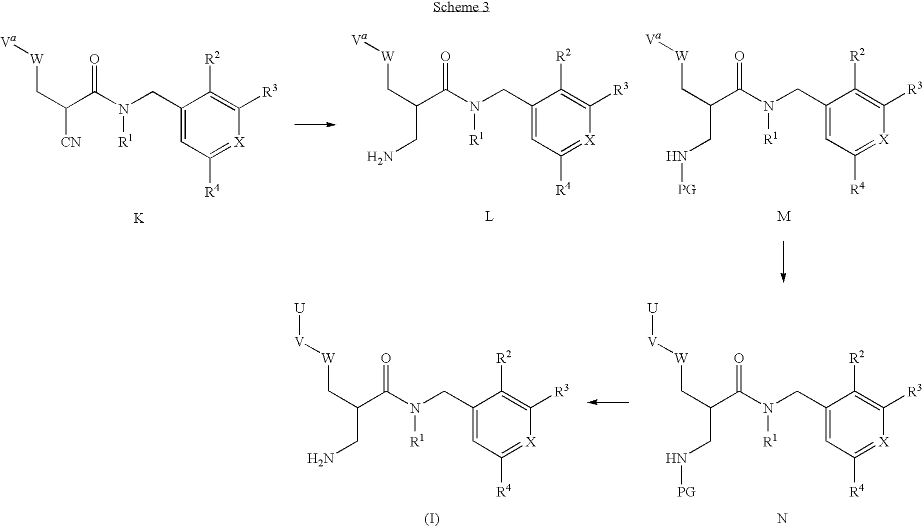

The U—V—W— or V^{a}—W-fragments that are used to prepare a compound of type A or F have to be prepared separately. The preparation of several such substituents is described in the patent applications WO 2003/093267, WO 2004/002957, WO 2004/096769, WO 2004/096803, WO 2004/096799, and WO 2004/096366. Otherwise a pyrrolidine substituent can be attached to an aromatic ring by a copper- or palladium-catalysed coupling as described in Scheme 4. Under certain circumstances a transition metal is not necessary to catalyse this reaction. A protected pyrrolidine derivative, wherein PG′ stands for a suitable protecting group, will be transformed into a compound of type F, wherein X′ stands for N. If W in formula (I) represents a thiazolyl, the same chemistry can be applied as well.

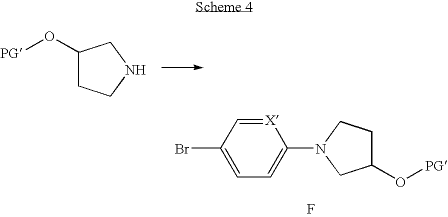

If V represents —O—CH₂-Q-, the isoxazolyl moiety is prepared by cycloaddition. This cycloaddition can be realized on the W—V^{a}-fragment in a compound of type K, leading to a compound of type E as described in Scheme 2. Otherwise the cycloaddition can be performed separately as, for instance, described in Scheme 5. Cycloaddition on a compound of type F with an often commercially available aldehyde leads to a compound of type A. Of course the aldehyde moiety can be built on the W—V^{a}-fragment, and a compound of the form U—CCH can be constructed, to give after cycloaddition another isoxazolyl moiety. The same principles can be used to prepare oxadiazolyl moieties, using methodologies described in the literature.

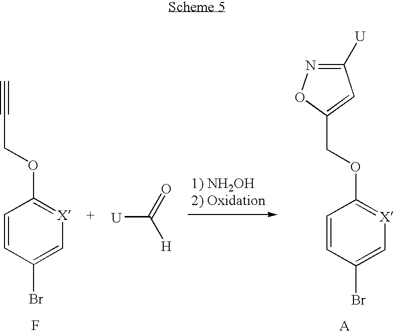

Also a hydroxymethyl isoxazole (Scheme 6) can be prepared from the aldehyde mentioned in Scheme 3 and propargyl alcohol. Coupling to a phenyl or heteroaryl derivative, wherein X″ typically stands for —OH, —Br, or —I, leads to a compound of type A.

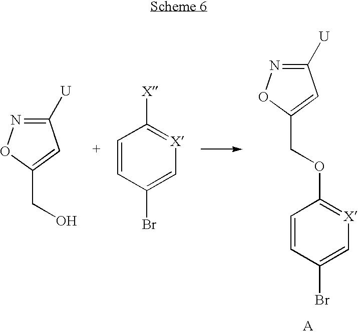

The amines used for such amide couplings have to be prepared separately, as described specifically in the examples, vide infra.

Enantiomerically pure compounds can always be obtained e.g. by chromatographic separation of the corresponding racemate, using a chiral solid support.

The following examples serve to illustrate the present invention in more details. They are, however, not intended to limit its scope in any manner.

### Experimental Part

### Abbreviations (as Used Herein):

AcOH acetic acid
ADDP azodicarboxylic dipiperidide
Ang angiotensin
aq. aqueous
Boc tert-butyloxycarbonyl
BSA bovine serum albumine
Bu butyl
BuLi n-butyllithium
Cy cyclohexyl
dba dibenzylidene acetone
DDQ 2,3-dichloro-5,6-dicyano-1,4-benzoquinone
DIPEA diisopropylethylamine
DMAP 4-N,N-dimethylaminopyridine
DMF N,N-dimethylformamide
DMSO dimethylsulfoxide
dppp 1,3-bis(diphenylphosphino)propane
EDC.HCl ethyl-N,N-dimethylaminopropylcarbodiimide hydrochloride
EIA enzyme immunoassay
ELSD evaporative light scattering detection
eq. equivalent(s)
ES electrospray
ES+ electrospray, positive ionization
Et ethyl
EtOAc ethyl acetate
EtOH ethanol
FC flash chromatography
h hour(s)
HATU O-(7-azabenzotriazol-1-yl)-N,N,N′,N′-tetramethyluronium hexafluorophosphate
HOBt hydroxybenzotriazol
HPLC high performance liquid chromatography
LC-MS liquid chromatography—mass spectrometry
Me methyl
MeOH methanol
min minute(s)
MS mass spectrometry
org. organic
p para
PG protecting group
rt room temperature
sat. saturated
sol. solution
TBAF tetra-n-butylammonium fluoride
TBME tert-butyl methyl ether
TBDMS tert-butyldimethylsilyl
tBu tert-butyl
TFA trifluoroacetic acid
THF tetrahydrofuran
TLC thin layer chromatography
t_{R} retention time (in LC-MS or HPLC) given in minutes
UV ultra violet
Vis visible
xantphos 4,5-bis(diphenylphosphino)-9,9-dimethylxanthene

### HPLC- or LC-MS-Conditions (if not Indicated Otherwise):

Analytic: Zorbax 59 SB Aqua column, 4.6×50 mm from Agilent Technologies. Eluents: A: acetonitrile; B: H₂O+0.5% TFA. Gradient: 90% B→5% B over 2 min. Flow: 1 mL/min. Detection: UV/Vis+MS.
Preparative: Zorbax SB Aqua column, 20×500 mm from Agilent Technologies. Eluent: A: Acetonitrile; B: H₂O+0.05% ammonium hydroxide (25% aq.). Gradient: 80% B→10% B over 6 min. Flow: 40 mL/min. Detection: UV+MS, or UV+ELSD.
Chiral, analytic:
a) Regis Whelk column, 4.6×250 mm, 10 μm. Eluent A: EtOH+0.05% Et₃N. Eluent B: hexane. Flow 1 mL/min.
b) ChiralPak AD, 4.6×250 mm, 5 μm. Eluent A: EtOH+0.05% Et₃N. Eluent B: hexane. Flow 1 mL/min.
c) ChiralCel OD, 4.6×250 mm, 10 μm. Eluent A: EtOH+0.1% Et₃N. Eluent B: hexane. Flow 0.8 mL/min.
   Chiral, preparative:
a) Regis Whelk 01 column, 50×250 mm and a flow of 100 mL/min. Eluent A: EtOH+0.05% Et₃N. Eluent B: hexane.
b) ChiralCel OD, 20 μm, 50 mm×250 mm, flow 100 mL/min. Eluent A: EtOH+0.1% Et₃N. Eluent B: hexane.

### General Conditions for a Mitsunobu Coupling (General Procedure A)

A mixture of the desired alcohol (0.1 mmol), of the desired phenol (0.12 mmol), ADDP (0.2 mmol) and PBu₃ (0.4 mmol) in toluene (2 mL) was prepared at 0° C. The mixture was stirred for 2 h at rt, for 3 h at reflux, and then overnight at rt again. The solvents were removed under reduced pressure, and the residue was purified by HPLC.

### General Conditions for the Cleavage of a Boc-Protecting Group (General Procedure B)

HCl (4M in dioxane, 1 mL) was added to a sol. of the starting material in CH₂Cl₂ (1 mL) at 0° C. The mixture was stirred for 1 h at 0° C., and aq. 1M NaOH (4 mL) was added. The mixture was filtered on Isolute®, and the org. layer was evaporated under reduced pressure. Purification of the crude by HPLC yielded the title compound.

### 2-Bromo-5-chloro-pyridine-4-carbaldehyde

To a stirred sol. of diisopropylamine (20.9 mL, 148 mmol) in dry THF (350 mL) at −5° C. was added dropwise BuLi (1.6M in hexane, 89.5 mL, 143 mmol), and the resulting sol. was stirred for 30 min at −5° C. The sol. was allowed to cool to −70° C., and a sol. of 2-bromo-5-chloropyridine (25.0 g, 130 mmol) in THF (100 mL) was added dropwise at −70° C. over 15 min, such that the internal temperature did not exceed −65° C. The mixture was stirred at −70° C. for 30 min. DMF (10.52 mL, 136 mmol) was added dropwise over 20 min such that the internal temperature did not exceed −70° C. The orange mixture was stirred at −70° C. for 40 min. The mixture was allowed to warm up to rt, and was poured onto a mixture of water (200 mL) and aq. 1M NaOH (50 mL). The mixture was extracted with EtOAc (2×), and the combined org. extracts were washed back with aq. 1M NaOH (2×). The org. extracts were dried over MgSO₄, filtered, and the solvents were removed under reduced pressure. Purification of the crude by FC (EtOAc/heptane 1:9→1:8→1:6→1:4→1:2→1:1) yielded the title compound (21.55 g, 72%). LC-MS: t_{R}=0.74 min; ES+: 295.01.

### 2-Bromo-5-chloro-4-dimethoxymethyl-pyridine

To a sol. of 2-bromo-5-chloro-pyridine-4-carbaldehyde (43.9 g, 199 mmol) in MeOH (800 mL) were successively added at rt trimethyl orthoformate (65.3 mL, 597 mmol) and p-toluenesulfonic acid monohydrate (1.90 g, 10.0 mmol). This reaction mixture was then heated to reflux for 3 h. The mixture was allowed to cool to rt and was concentrated under reduced pressure. The residue was dissolved in CH₂Cl₂ and this mixture was washed with aq. 10% K₂CO₃. The org. layer was dried over MgSO₄, filtered, and the solvents were removed under reduced pressure. Drying under high vacuum yielded the title compound (51.7 g, 97%). LC-MS: t_{R}=0.92 min; ES+: 309.06.

### 5-Chloro-4-dimethoxymethyl-2-(3-methoxy-propyl)-pyridine

To a suspension of Mg (911 mg, 37.5 mmol) and of iodine (one crystal) in dry THF (30 mL) was added dropwise 5% of the total amount of 1-bromo-3-methoxypropane (4.59 g, 30.0 mmol). The mixture was heated to reflux with the help of a heat gun until the Grignard formation had started. The rest of the 1-bromo-3-methoxypropane was added slowly, while an exothermic reaction proceeded. After the end of the addition, the reaction mixture was stirred under reflux for 20 min, and was allowed to cool to rt. This Grignard sol. (1M in THF, 23.5 mL, 23.5 mmol) was added dropwise to a mixture of 2-bromo-5-chloro-4-dimethoxymethyl-pyridine (2.50 g, 9.38 mmol) and Ni(dppp)Cl₂ (495 mg, 0.938 mmol) in THF (50 mL) at 0° C. The reaction mixture was stirred at rt for 30 min, and was then heated to reflux for 2 h. The mixture was allowed to cool to rt, and was dissolved with EtOAc. This mixture was washed with aq. sat. NaHCO₃. The org. layer was dried over MgSO₄, filtered, and the solvents were removed under reduced pressure. Purification of the residue by FC (heptane o EtOAc/heptane 1:1) yielded the title compound (1.51 g, 62%). LC-MS: t_{R}=0.80 min; ES+: 260.15.

### 5-Chloro-2-(3-methoxy-propyl)-pyridine-4-carbaldehyde

5-Chloro-4-dimethoxymethyl-2-(3-methoxy-propyl)-pyridine (25.5 g, 98.2 mmol) was dissolved in aq. 1M HCl (500 mL), and the mixture was heated to 80° C. for 2 h. The mixture was allowed to cool to rt, and EtOAc was added. The mixture was cooled to 0° C., and was basified with aq. 2.5M NaOH until a pH=10 was reached. The layers were separated, and the org. layer was dried over MgSO₄, filtered, and concentrated under reduced pressure. Drying the residue under high vacuum yielded the crude title compound (98.1 mmol, 99%) that was used further without purification. LC-MS: t_{R}=0.62 min; ES+: 246.12.

### [5-Chloro-2-(3-methoxy-propyl)-pyridin-4-ylmethyl]-cyclopropyl-amine

A mixture of 5-chloro-2-(3-methoxy-propyl)-pyridine-4-carbaldehyde (21.0 g, 98.2 mmol) and cyclopropylamine (13.8 mL, 196 mmol) in MeOH (450 mL) was stirred at rt overnight. NaBH₄ (4.83 g, 128 mmol) was added at 0° C., and the mixture was stirred at rt overnight. Ice was added, and the mixture was concentrated under reduced pressure. The crude product was dissolved in EtOAc, and this mixture was washed with aq. 1M NaOH. The aq. layer was extracted back with EtOAc. The combined org. extracts were dried over MgSO₄, filtered, and the solvents were removed under reduced pressure. Purification of the crude by FC (EtOAc/heptane 1:5→1:4→1:3→1:1→3:1→EtOAc) yielded the title compound (11.8 g) and [5-chloro-2-(3-methoxy-propyl)-pyridin-4-ylmethylene]-cyclopropyl-amine (10.7 g). This unreacted inine was dissolved in MeOH (20 mL), and this sol. was cooled to 0° C. NaBH₄ (3.20 g, 84.6 mmol) was added, and the mixture was stirred at rt overnight. NaBH₄ (3.20 g, 84.6 mmol) was added again, and the mixture was stirred for 3 days. Ice was added to the reaction mixture, and the mixture was concentrated under reduced pressure. The crude product was dissolved in EtOAc and the resulting mixture was washed with aq. 1M NaOH. The aq. phase was extracted back with EtOAc. The combined org. extracts were dried over MgSO₄, filtered, and the solvents were removed under reduced pressure. Purification of the crude by FC (EtOAc/heptane 1:3→1:2→1:1→EtOAc) yielded the title compound (9.4 g). The fractions of the title compound were mixed together (21.2 g, 85%). LC-MS: t_{R}=0.55 min; ES+: 296.16.

### 5-Bromo-2-chloro-N-cyclopropylbenzamide

Into a flame-dried 250 mL round-bottom flask equipped with a magnetic stir bar and under N₂ were added 5-bromo-2-chlorobenzoic acid (10.0 g, 42.5 mmol) and DMF (3.9 mL, 51.0 mmol) in toluene (80 mL). The sol. was cooled to 0° C., and oxalyl chloride (4.4 mL, 51.0 mmol) was added dropwise over 1 h. The resulting mixture was stirred at 0° C. for 2 h and then the volatiles were removed. The resulting crude reaction mixture was dissolved in CH₂Cl₂ (100 mL) and cooled to 0° C. in an ice bath. Cyclopropylamine (4.5 mL, 63.7 mmol) was added dropwise over 1 h followed by addition of DIPEA (11.8 mL, 85.0 mmol). The resulting sol. was stirred at rt for 16 h. The reaction mixture was poured into a 1 L separatory funnel containing 1M aq. HCl (600 mL). The mixture was extracted with CH₂Cl₂ (6×250 mL). The combined org. layers were washed with brine, dried over MgSO₄, filtered and concentrated under reduced pressure. The product was crystallized from hexanes/CH₂Cl₂ and isolated by filtration to give the title compound (8.24 g, 71%).

### N-(5-bromo-2-chlorobenzyl)cyclopropylamine

A sol. of 5-bromo-2-chloro-N-cyclopropylbenzamide (12.0 g, 43.7 mmol) in THF (100 mL) was placed into a 250 mL round-bottom flask, equipped with a magnetic stir bar and under N₂. The sol. was treated with dropwise addition of BH₃.Me₂S (13.1 mL, 131 mmol), and the resulting suspension was stirred at rt for 1 h. The mixture was heated to reflux for 1 h, cooled to rt, and slowly quenched with dropwise addition of 1M aq. HCl (25 mL). The suspension was again refluxed for 1 h, cooled to rt, and basified to pH=10-11 with 1M aq. NaOH. The mixture was poured into a 500 mL separatory funnel containing 1M aq. NaOH (350 mL). The mixture was extracted with EtOAc (3×100 mL). The combined org. layers were washed with brine, dried over MgSO₄, filtered and concentrated under reduced pressure. The crude amine was used directly in the next step.

General Procedure for the Reductive Amination of Substituted Benzaldehydes with Cyclopropylamine:

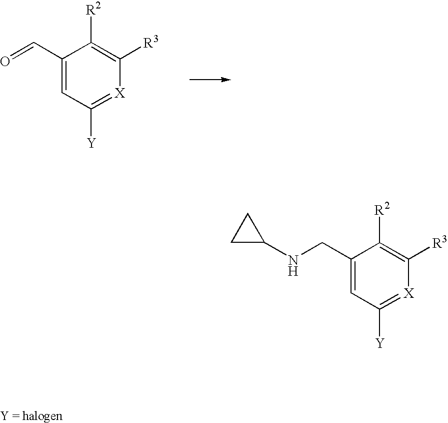

A sol. of substituted benzaldehyde (17.8 mmol, 1.0 eq.), cyclopropylamine (3.13 mL, 44.5 mmol, 2.5 eq.) and sodium cyanoborohydride (1.34 g, 21.4 mmol, 1.2 eq.) in MeOH (100 mL) was treated with dropwise addition of glacial AcOH (3.06 mL, 53.4 mmol, 3.0 eq.). The resulting sol. was stirred at rt for 16 h overnight. The reaction mixture was quenched with dropwise addition of sat. aq. NaHCO₃, and concentrated under reduced pressure to remove the MeOH. The crude residue was poured into a 250 mL separatory funnel containing sat. aq. NaHCO₃ (150 mL), and extracted with EtOAc (3×50 mL). The combined org. layers were washed with brine, dried over MgSO₄, filtered and concentrated under reduced pressure. Purification by FC yielded the benzamine product.

### General Procedure for the Boc-Protection of Cyclopropylbenzamines:

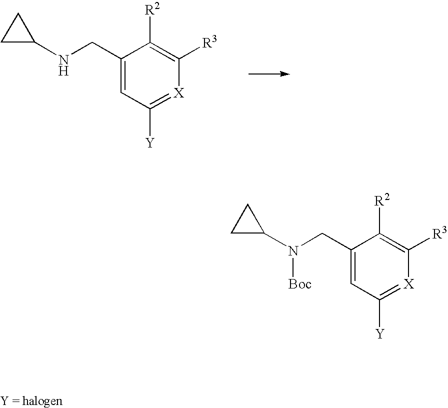

A sol. of the cyclopropylbenzamine (43.7 mmol, 1.0 eq.) in a biphasic mixture of CH₂Cl₂ (50 mL) and 1M aq. NaOH (50 mL) was treated with Boc₂O (15.1 mL, 65.6 mmol, 1.5 eq.). The mixture was stirred at rt vigorously for 16 h. The mixture was poured into a 500 mL separatory funnel containing H₂O (300 mL), and extracted with CH₂Cl₂ (3×100 mL). The combined org. layers were washed with brine, dried over MgSO₄, filtered and concentrated under reduced pressure. Purification by FC yielded the Boc-protected amine.

### General Procedure for the Allylation of Boc-Protected Cyclopropylbenzamines:

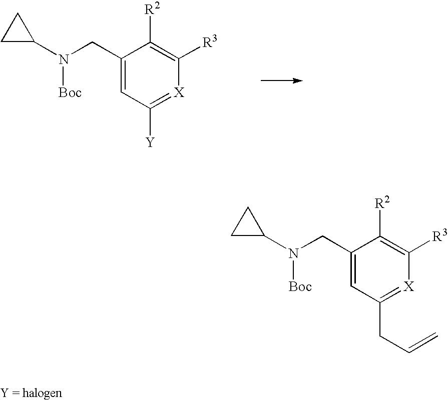

Into a flame-dried round-bottom flask or Schlenk tube, under N₂ was added Pd[PCy₃]₂ (0.05 eq.), CsF (2.0 eq.) and the corresponding aryl bromide (1.0 eq.). If the aryl chloride was being used as a starting material, the (Pd[PtBu₃]Br)₂ dimer (0.025 eq.) was used in place of the Pd[PCy₃]₂ catalyst. The flask was evacuated under reduced pressure (0.1 mm Hg) and backfilled with N₂ (repeated 3 times). The resulting solids were dissolved in anhydrous THF or dioxane (0.15 M sol.) and tri-n-butyl allyltin (1.5 eq.) was added and the resulting mixture was refluxed for 8-16 h, until TLC shows complete consumption of starting material. The reaction mixture was cooled to rt, and filtered through a pad of silica gel on a sintered glass funnel, washing with Et₂O. The filtrate was concentrated and purified by FC to give the corresponding allylbenzamide derivative.

### General Procedure for the Hydroboration/Oxidation of Allylbenzamines:

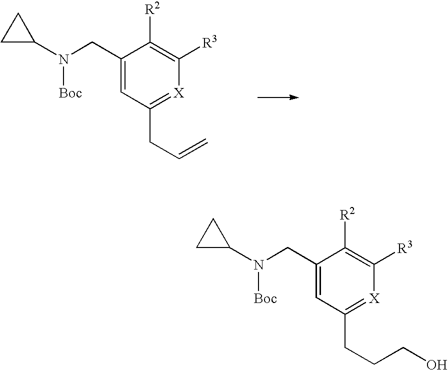

Into a flame-dried round-bottom flask equipped with a magnetic stir bar was added the allylbenzamine (1.0 eq.) and anhydrous THF (0.3 M sol.). The sol. was cooled to 0° C. and BH₃.Me₂S (1.1 eq.) was added dropwise over 20 min. The sol. was stirred at 0° C. for 1 h, then allowed to warm to rt, and stirred for an additional 2 h. The sol. was cooled to 0° C. and 1M aq. NaOH was added dropwise (CAUTION—EXOTHERMIC REACTION), followed by dropwise addition of 30% aq. H₂O₂. The mixture was allowed to warm to rt, and stirred for 2 h. The mixture was poured into a separatory funnel containing H₂O and extracted with Et₂O (3 times). The combined org. layers were washed with brine, dried over MgSO₄, filtered and concentrated under reduced pressure. Purification by FC yielded the desired alcohol product.

### General Procedure for the Oxidative Cleavage/Reduction of Allylbenzamines:

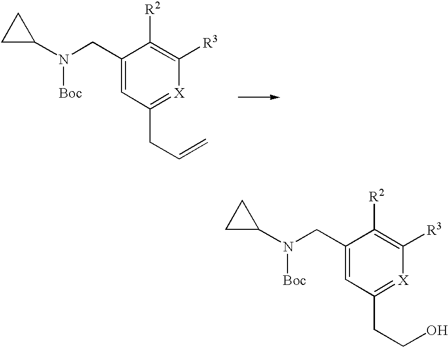

A sol. of allylbenzamine (1.0 eq.) in CH₂Cl₂ (0.4 M sol.) was cooled to −78° C. and O₃ gas was introduced into the sol. using a gas dispersion tube. The ozone gas was introduced until all of the starting material had been consumed, as determined by TLC, and the reaction mixture maintained a slight blue colour. The reaction was stirred at −78° C. for 20 min, then EtOH (0.5 M sol.) and NaBH₄ (2.5 eq.) were added. The mixture was allowed to warm to rt overnight (16 h). The reaction mixture was quenched with dropwise addition of sat. aq. NH₄Cl (5 mL), and poured into a separatory funnel containing sat. aq. NH₄Cl. The mixture was extracted with Et₂O (3 times). The combined org. layers were washed with brine, dried over MgSO₄, filtered and concentrated under reduced pressure. Purification by FC yielded the desired alcohol.

General Procedure for the Etherification of Aromatic Primary Alcohols with Methyl Iodide:

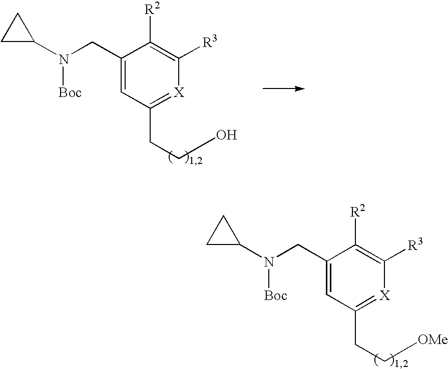

A suspension of the primary alcohol (1.0 eq.) in THF (0.25 M sol.) was cooled to 0° C. and treated with NaH (60% in oil, 2.0 eq.). The resulting mixture was stirred at 0° C. for 30 min and then at rt for another 30 min. The suspension was re-cooled to 0° C. and then MeI (8.0 eq.) was added in a single portion. The reaction mixture was stirred at 0° C. for 30 min, at rt for 30 min, and then heated to reflux for 4 h until all of the starting material was consumed as determined by TLC. The cooled reaction mixture was quenched with dropwise addition of sat. aq. NH₄Cl and poured into a separatory funnel containing sat. aq. NH₄Cl, and extracted with EtOAc (3 times). The combined org. layers were washed with brine, dried over MgSO₄, filtered and concentrated under reduced pressure. Purification by FC yielded the methyl ether.

### General Procedure for the Deprotection of Boc-Protected Cyclopropylbenzamines:

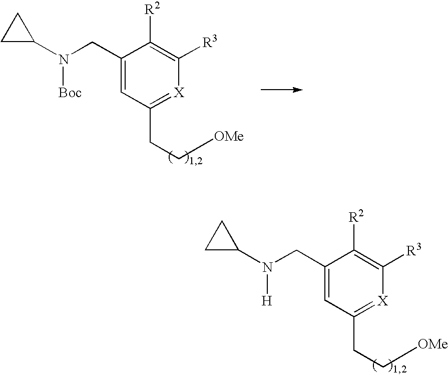

To a sol. of Boc-protected cyclopropylbenzamine (1.0 eq.) in CH₂Cl₂ (0.1-0.5 M sol.) was added 4 M HCl in dioxane (5.0 eq.). The resulting mixture was stirred at rt for 8-16 h until TLC shows complete conversion of starting material. The reaction was poured into a separatory funnel containing 1M aq. NaOH, and extracted with CH₂Cl₂ (3 times). Purification by FC yielded the corresponding free amine.

### 2,6-Dichloro-4-hydroxymethylphenol

BH₃ (1M in THF, 250 mL, 250 mmol) was added dropwise to a cooled sol. of 3,5-dichloro-4-hydroxybenzoic acid (20 g, 96.6 mmol) in THF (200 mL) at 0° C. The resulting mixture was stirred at 0° C. for 15 min, and then at rt for 13 h. The milky mixture was cooled to 0° C. and MeOH (150 mL), then water (100 mL), were added dropwise. The mixture was further stirred at 0° C. for 15 min, and then at rt for 5 h. The mixture was then partially concentrated under reduced pressure. EtOAc (200 mL) and water (50 mL) were added to the residue, and the phases were shaken and separated. The aq. phase was further extracted with EtOAc. The combined org. extracts were washed with brine, dried over MgSO₄, filtered, and concentrated under reduced pressure. Purification by FC (CH₂Cl₂/CH₃OH, 100:1) led to the title compound as a slightly beige solid (17.86 g, 96%). LC-MS: t_{R}=0.69 min.

### 3,5-Dichloro-4-hydroxybenzaldehyde

2,6-Dichloro-4-hydroxymethylphenol (3.56 g, 18.4 mmol) was dissolved in dioxane, and DDQ (4.19 g, 18.4 mmol) was added. The reaction mixture was stirred at rt overnight. The solvents were removed under reduced pressure. The residue was diluted with CH₂Cl₂, and the mixture was filtered. The filtrate was dried over MgSO₄, filtered, and the solvents were removed under reduced pressure. Crystallization from EtOAc yielded the title compound (0.77 g, 22%). LC-MS: t_{R}=0.82 min.

### (rac.)-2,6-Dichloro-4-(1-hydroxyethyl)phenol

A sol. of 3,5-dichloro-4-hydroxybenzaldehyde (1.635 g, 8.56 mmol) in Et₂O (30 mL) was cooled to −78° C. MeMgBr (3M in Et₂O, 7.15 mL, 21.5 mmol) was added dropwise to the cooled reaction mixture over 18 min. Et₂O (20 mL) was added again during the addition of MeMgBr. Stirring was continued at −78° C. for 1 h, and then the reaction mixture was allowed to warm up to rt over 1 h. The mixture was cooled to 0° C., and aq. sat. NH₄Cl (10 mL) was added dropwise. The mixture was allowed to warm up to rt, and additional aq. sat. NH₄Cl (35 mL) and water (35 mL) were added. The phases were then separated and the aq. phase was extracted with Et₂O. The combined org. extracts were then washed with brine (50 mL), dried over MgSO₄, filtered, and concentrated under reduced pressure. Purification by FC (EtOAc/heptane, 1:1) yielded the title compound (1.68 g, 95%). LC-MS: t_{R}=0.74 min.

### (rac)-2-(tert-Butyldimethylsilanyloxy)-5-[1-(tert-butyldimethylsilanyloxy)ethyl]-1,3-dichlorobenzene

To a sol. of (rac.)-2,6-dichloro-4-(1-hydroxyethyl)phenol (100 mg, 0.483 mmol) in DMF (5.5 mL) were added TBDMS-Cl (175 mg, 1.16 mmol), and imidazole (145 mg, 2.42 mmol). The sol. was stirred at rt overnight. The sol. was cooled to 0° C., and aq. sat. NH₄Cl was added. The mixture was extracted with heptane/Et₂O (1/1, 4×). The combined org. extracts were dried over MgSO₄, filtered, and the solvents were removed under reduced pressure. Purification by FC(CH₂Cl₂) yielded the title compound (188 mg, (90%).

### (rac.)-4-[1-(tert-Butyldimethylsilanyloxy)ethyl]-2,6-dichlorophenol

A sol. of (rac.)-2-(tert-butyldimethylsilanyloxy)-5-[1-(tert-butyldimethylsilanyloxy)ethyl]-1,3-dichlorobenzene (188 mg, 0.432 mmol) and Cs₂CO₃ (76.2 mg, 0.126 mmol) in DMF (0.50 mL) and water (50 μL) was stirred at rt overnight. Et₂O (75 mL) was added. The sol. was washed with brine, dried over MgSO₄, filtered, and the solvents were removed under reduced pressure. Purification by FC(CH₂Cl₂) yielded the title compound (122 mg, 88%). LC-MS: t_{R}=1.15 min.

### 2,6-Dichloro-3,4-dimethylphenol

To a sol. of 3,4-dimethylphenol (3.00 g, 24.6 mmol) in CH₂Cl₂ (5 mL) was added SO₂Cl₂ (4.98 mL, 61.3 mmol). The resulting sol. was heated to 50° C. for 4 h. The mixture was poured onto ice-water. CH₂Cl₂ (200 mL) was added, the layers were separated, and the org. layer was washed with water, then with aq. sat. NaHCO₃. The org. layer was dried over MgSO₄, filtered, and the solvents were removed under reduced pressure. Purification by FC (EtOAc/heptane 1:4) yielded the title compound (1.17 g, 25%).

### 2-[2-(tert-Butyldimethylsilanyloxy)ethoxy]thiazole

NaH (50% suspension in oil, 2.98 g, 62.1 mmol) was suspended in hexane and washed twice with hexane. THF (20 mL) was then added followed by a sol. of 2-(tert-butyldimethylsilanyloxy)ethanol (McDougal, P. G., Rico, J. G., Oh, Y. I., Condon, B. D., J. Org. Chem., 1986, 51, 3388, 9.49 g, 53.8 mmol) in THF (30 mL) over 30 min. The mixture was then stirred for 2 h at rt. 2-Bromothiazole (6.79 g, 41.4 mmol) was then added dropwise and the reaction mixture was then stirred at reflux for 20 h. Aq. sat. NH₄Cl was added carefully and the product was extracted with Et₂O (3×). The combined org. extracts were dried over MgSO₄, filtered, and the solvents were removed under reduced pressure. Purification of the residue by FC (Et₂O/hexane 5:95) yielded the title compound (3.80 g, 35%).

### (S)-1-Thiazol-2-yl-pyrrolidin-3-ol

A mixture of 2,5-dibromothiazole (15.0 g, 59.9 mmol), (S)-3-hydroxypyrrolidine (6.00 mL, 71.9 mmol) and DIPEA (13.3 mL, 77.9 mmol) in dioxane (875 mL) was stirred at 80° C. for 17 h. The solvents were removed under reduced pressure. Aq. sat. NaHCO₃ was added, and the mixture was extracted with CH₂Cl₂ (2×). The combined org. extracts were dried over MgSO₄, filtered, and the solvents were removed under reduced pressure. The crude title product (12.2 g) was used in the next reaction without purification. LC-MS: t_{R}=0.54 min; ES+: 249.06.

### (S)-2-[3-(tert-Butyl-dimethyl-silanyloxy)-pyrrolidin-1-yl]-thiazole

A mixture of (S)-1-thiazol-2-yl-pyrrolidin-3-ol (12.2 g, 48.9 mmol), TBDMS-Cl (8.84 g, 58.7 mmol) and imidazole (8.30 g, 122 mmol) in DMF (150 mL) was stirred at rt for 30 min. Water (150 mL) was added, and the mixture was extracted with heptane (3×). The combined org. extracts were dried over MgSO₄, filtered, and the solvents were removed under reduced pressure. Purification of the residue by FC (heptane/EtOAc 14:1→13:1→12:1→10:1) yielded the title compound (11.3 g, 52% over 2 steps). LC-MS: t_{R}=1.10 min; ES+: 363.14.

### 2-Cyano-N-cyclopropyl-N-(2,3-dimethyl-benzyl)-acetamide

HATU (6.51 g, 17.1 mmol) was added to a sol. of cyano-acetic acid (1.46 g, 17.1 mmol), cyclopropyl-(2,3-dimethyl-benzyl)-amine (prepared from 2,3-dimethyl-benzaldehyde and cyclopropylamine by reductive amination; 3.00 g, 17.1 mmol) and DIPEA (5.86 mL, 34.2 mmol) in DMF (15 mL) at 0° C. The mixture was stirred for 1.5 h at 0° C., and was diluted with EtOAc. The resulting mixture was washed with aq. 1M HCl (2×) and aq. sat. NaHCO₃ (1×). The combined aq. phases were extracted back with EtOAc (1×). The combined org. extracts were dried over MgSO₄, filtered, and the solvents were removed under reduced pressure. Purification of the residue by FC (EtOAc/heptane 1:3→1:2→1:1) yielded the title compound (3.36 g, 81%). LC-MS: t_{R}=0.93 min; ES+: 243.22.

### 2-Cyano-N-cyclopropyl-N-(2,3-dichloro-benzyl)-acetamide

HATU (4.47 g, 11.8 mmol) was added to a sol. of cyano-acetic acid (1.00 g, 11.8 mmol), cyclopropyl-(2,3-dichloro-benzyl)-amine (prepared from 2,3-dichloro-benzaldehyde and cyclopropylamine by reductive amination; 2.54 g, 11.8 mmol) and DIPEA (4.03 mL, 23.5 mmol) in DMF (10 mL) at 0° C. The mixture was stirred for 1 h at 0° C., and for 1.5 h at rt. EtOAc was added, and the mixture was washed with aq. 1M HCl (2×), and aq. sat. NaHCO₃ (1×). The combined aq. phases were extracted back with EtOAc (1×). The combined org. extracts were dried over MgSO₄, filtered, and the solvents were removed under reduced pressure. Purification of the crude by FC (EtOAc/heptane 1:1) yielded the title compound (2.58 g, 78%). LC-MS: t_{R}=0.94 min; ES+: 324.09.

### (E)-3-{2-[2-(tert-Butyl-dimethyl-silanyloxy)-ethoxy]-thiazol-5-yl}-2-cyano-N-cyclopropyl-N-(2,3-dimethyl-benzyl)-acrylamide

A mixture of 2-cyano-N-cyclopropyl-N-(2,3-dimethyl-benzyl)-acetamide (3.36 g, 13.9 mmol), compound B1 (3.99 g, 13.9 mmol) and pyridine (5 droplets) in toluene (100 mL) was heated to 120° C. for 2 days. The mixture was allowed to cool to rt, and was evaporated under reduced pressure. The residue was dried under high vacuum. The resulting yellow crystals were triturated with heptane, and were filtered. Drying these crystals under high vacuum yielded the title compound (4.86 g, 69%). LC-MS: t_{R}=1.21 min; ES+: 512.37.

### (E)-(S)-3-{2-[3-(tert-Butyl-dimethyl-silanyloxy)-pyrrolidin-1-yl]-thiazol-5-yl}-2-cyano-N-cyclopropyl-N-(2,3-dichloro-benzyl)-acrylamide

A mixture of 2-cyano-N-cyclopropyl-N-(2,3-dichloro-benzyl)-acetamide (2.13 g, 7.52 mmol), compound B2 (2.35 g, 7.52 mmol) and pyridine (5 droplets) in toluene (100 mL) was heated to 120° C. for 2 days. The mixture was allowed to cool to rt, and the solvents were removed under reduced pressure. The residue was dried under high vacuum, and was dissolved in a mixture of EtOAc and heptane. The solvents were removed under reduced pressure, and the resulting solid was triturated with heptane. Drying the solid under high vacuum yielded the title compound (3.76 g, 86%). LC-MS: t_{R}=1.24 min; ES+: 577.02.

### (rac.)-2-{2-[2-(tert-Butyl-dimethyl-silanyloxy)-ethoxy]-thiazol-5-ylmethyl}-2-cyano-N-cyclopropyl-N-(2,3-dimethyl-benzyl)-acetamide

(E)-3-{2-[2-(tert-Butyl-dimethyl-silanyloxy)-ethoxy]-thiazol-5-yl}-2-cyano-N-cyclopropyl-N-(2,3-dimethyl-benzyl)-acrylamide (4.17 g, 8.15 mmol) was suspended in MeOH (50 mL), and THF was added until a clear sol. was obtained (about 80 mL). The mixture was cooled to 0° C., and CeCl₃.7H₂O (6.20 g, 16.3 mmol) was added. NaBH₄ (1.61 g, 40.7 mmol) was added carefully in portions. The mixture was stirred for 30 min at 0° C., and CH₂Cl₂ (100 mL) was added. Aq. 1M NaOH was added, and the mixture was stirred efficiently for 10 min. The phases were separated, and the aq. phase was extracted with CH₂Cl₂. The combined org. extracts were washed with brine, dried over MgSO₄, filtered, and the solvents were removed under reduced pressure. Drying the residue under high vacuum yielded the title crude compound (4.47 g, quantitative) that was used further without purification. LC-MS: t_{R}=1.20 min; ES+: 514.43.

### Mixture of (2R)-3-{2-[(3S)-3-(tert-Butyl-dimethyl-silanyloxy)-pyrrolidin-1-yl]-thiazol-5-yl}-2-cyano-N-cyclopropyl-N-(2,3-dichloro-benzyl)-propionamide and (2S)-3-{2-[(3S)-3-(tert-Butyl-dimethyl-silanyloxy)-pyrrolidin-1-yl]-thiazol-5-yl}-2-cyano-N-cyclopropyl-N-(2,3-dichloro-benzyl)-propionamide

(E)-(S)-3-{2-[3-(tert-Butyl-dimethyl-silanyloxy)-pyrrolidin-1-yl]-thiazol-5-yl}-2-cyano-N-cyclopropyl-N-(2,3-dichloro-benzyl)-acrylamide (4.35 g, 7.53 mmol) was suspended in MeOH (65 mL), and THF was added until a clear sol. was obtained (about 30 mL). The mixture was cooled to 0° C., and CeCl₃.7H₂O (5.70 g, 15.1 mmol) was added. NaBH₄ (2.97 g, 75.3 mmol) was added carefully in portions. The mixture was stirred for 2 h at 0° C. CH₂Cl₂ (300 mL), MeOH (15 mL), and aq. 1M NaOH (200 mL) were added, and the mixture was stirred efficiently for 2 h. The phases were separated, and the org. layer was washed with aq. 1M NaOH and brine. The org. layer was dried over MgSO₄, filtered, and the solvents were removed under reduced pressure. Drying the residue under high vacuum yielded the crude title compounds mixture (4.50 g, quantitative yield) that was used without further purification. LC-MS: t_{R}=1.01 min; ES+: 579.20.

### 2-(2,6-Dichloro-4-methyl-phenoxy)-ethanol

In a three-necked flask equipped with a gas droplet counter and an efficient cooling system, a mixture of 2,6-dichloro-p-cresol (20.0 g, 113 mmol), [1,3]dioxolan-2-one (9.95 g, 113 mmol) and imidazole (115 mg, 1.70 mmol) was heated to 160° C. for 25 h. The mixture was allowed to cool to rt. Purification by FC (Et₂O/heptane 1:1) yielded the title compound (18.7 g, 75%). LC-MS: t_{R}=0.88 min.

### (R)-1-(5-Bromo-pyridin-2-yl)-pyrrolidin-3-ol

2,5-Dibromopyridine (4.00 g, 16.4 mmol) and (R)-pyrrolidine-3-ol (1.11 g, 12.6 mmol) were dissolved in toluene. tBuONa (1.87 g, 18.9 mmol), Pd₂(dba)₃ (231 mg, 0.252 mmol) and xantphos (451 mg, 0.756 mmol) were added, and the mixture was degassed with nitrogen. The mixture was stirred at 95° C. for 4 h, and was allowed to cool to rt. EtOAc was added, and the mixture was washed with water (2×). The combined org. layers were extracted back with EtOAc (1×). The combined org. extracts were dried over MgSO₄, filtered, and the solvents were removed under reduced pressure. Purification of the crude by FC (MeOH/CH₂Cl₂ 1:30 with 1% Et₃N) yielded the title compound (2.57 g, 84%). LC-MS: t_{R}=0.44 min; ES+: 243.07.

### (R)-5-Bromo-2-[3-(2,6-dichloro-4-methyl-phenoxy)-pyrrolidin-1-yl]-pyridine (A1)

A mixture of compound F1 (3.38 g, 13.9 mmol), 2,6-dichloro-p-cresol (3.69 g, 20.9 mmol), ADDP (5.26 g, 20.9 mmol) and PBu₃ (85%, 10.3 mL, 35.4 mmol) in toluene (200 mL) was heated to reflux for 2 h. The mixture was allowed to cool to rt, and was diluted with heptane. The mixture was filtered, washed with heptane, and the filtrate was evaporated under reduced pressure. The residue was diluted with CH₂Cl₂, and this mixture was washed with aq. 1M NaOH (2×). The org. layer was dried over MgSO₄, filtered, and the solvents were removed under reduced pressure. Purification of the residue by FC (CH₂Cl₂/heptane 4:1→CH₂Cl₂) yielded the title compound (5.46 g, 98%). LC-MS: t_{R}=0.91 min; ES+: 403.00.

### 5-Bromo-2-[2-(2,6-dichloro-4-methyl-phenoxy)-ethoxy]-pyridine (A2)

A sol. of 2-(2,6-dichloro-4-methyl-phenoxy)-ethanol (18.6 g, 84 mmol) in THF (360 mL) was cooled to 0° C. NaH (about 55% in oil, 6.60 g, about 153 mmol) was added in portions, and the mixture was stirred at rt for 30 min. A sol. of 2,5-dibrompyridine (18.0 g, 76.3 mmol) in THF (60 mL) was added dropwise, and the mixture was heated to reflux for 90 min. The mixture was allowed to cool to rt, and ice was added carefully. The solvents were partially removed under reduced pressure, and the residue was diluted with EtOAc. This mixture was washed with aq. sat. NH₄Cl. The aq. layer was extracted back with EtOAc (2×). The combined org. extracts were washed with brine, dried over MgSO₄, filtered, and the solvents were removed under reduced pressure. Purification of the crude by FC (EtOAc/heptane 3:97) yielded the title compound (22.7 g, 79%). LC-MS: t_{R}=1.13 min; ES+: 378.08.

### (S)-5-Bromo-2-[3-(2,6-dichloro-4-methyl-phenoxy)pyrrolidin-1-yl]-pyridine (A3)

A mixture of (R)-1-(5-bromo-pyridin-2-yl)-pyrrolidin-3-ol (2.56 g, 10.5 mmol), 2,6-dichloro-p-cresol (3.88 g, 21.1 mmol), ADDP (4.07 g, 15.8 mmol) and PBu₃ (85%, 9.17 mL, 26.8 mmol) in toluene (150 mL) was stirred at 80° C. for 1 h. The mixture was allowed to cool to rt, and was diluted with heptane. The precipitate was washed with heptane thoroughly, and the filtrate was evaporated under reduced pressure. The residue was diluted with CH₂Cl₂, and the resulting mixture was washed with aq. 1M NaOH (2×). The org. layer was dried over MgSO₄, filtered, and the solvents were removed under reduced pressure. Purification of the crude by FC (EtOAc/heptane 1:7) yielded the title compound (2.88 g, 68%). LC-MS: t_{R}=0.93 min; ES+: 402.89.

### 2-[2-(tert-Butyl-dimethyl-silanyloxy)-ethoxy]-thiazole-5-carbaldehyde (B1)

BuLi (9.95 mL, 15.6 mmol) was added to a sol. of 2-[2-(tert-butyldimethylsilanyloxy)ethoxy]thiazole (4.00 g, 15.4 mmol) in THF (25 mL) at −78° C. DMF (1.29 mL, 16.7 mmol) was added, and the mixture was stirred for 4 h, while being allowed to warm up to rt. Water was added, and the mixture was extracted with EtOAc (2×). The combined org. extracts were washed with brine (1×), dried over MgSO₄, filtered, and the solvents were removed under reduced pressure. Drying the residue under high vacuum yielded the crude title compound (4.35 g, 98%) that was used further without purification. LC-MS: t_{R}=1.08 min; ES+: 288.22.

### (S)-2-[3-(tert-Butyl-dimethyl-silanyloxy)-pyrrolidin-1-yl]-thiazole-5-carbaldehyde (B2)

BuLi (1.6M in hexane, 2.27 mL, 3.63 mmol) was added to a sol. of (S)-2-[3-(tert-butyl-dimethyl-silanyloxy)-pyrrolidin-1-yl]-thiazole (1.00 g, 3.52 mmol) in THF (6.00 mL) at −78° C. The mixture was stirred for 10 min at −78° C., and DMF (0.294 mL, 3.81 mmol) was added. The mixture was stirred for 2 h while being allowed to warm up to rt. Water was added, and the mixture was extracted with EtOAc (2×). The combined org. extracts were dried over MgSO₄, filtered, and the solvents were removed under reduced pressure. The crude title compound (1.10 g, quantitative yield) was used without further purification. LC-MS: t_{R}=1.08 min; ES+: 313.18.

### (R)-6-[3-(2,6-Dichloro-4-methyl-phenoxy)-pyrrolidin-1-yl]-pyridine-3-carbaldehyde (B3)

BuLi (1.6M in hexane, 9.80 mL, 16.3 mmol) was added to a sol. of compound A1 (5.46 g, 13.6 mmol) in THF (280 mL) at −78° C. The mixture was stirred for 20 min at −78° C., and DMF (1.57 mL, 20.3 mmol) was added. The mixture was stirred for 2.5 h at −78° C., and aq. sat. NH₄Cl (120 mL) was added. The mixture was allowed to warm up to rt, and was extracted with TBME (2×). The combined org. extracts were washed with aq. sat. NH₄Cl, dried over MgSO₄, filtered, and the solvents were removed under reduced pressure. Purification of the crude by FC (EtOAc/heptane 1:4→2:3) yielded the title compound (2.97 g, 62%). LC-MS: t_{R}=0.89 min; ES+: 351.12.

### 6-[2-(2,6-Dichloro-4-methyl-phenoxy)-ethoxy]-pyridine-3-carbaldehyde (B4)

BuLi (1.6M in hexane, 9.53 mL, 15.3 mmol) was added to a sol. of compound A2 (5.00 g, 13.3 mmol) in THF (280 mL) at −78° C. The mixture was stirred for 1 h at −78° C., and DMF (1.54 mL, 19.9 mmol) was added. The mixture was stirred for 2.5 h at −78° C., and DMF (1.00 mL, 12.9 mmol) was added again. The mixture was stirred overnight while warming up to rt. Aq. sat. NH₄Cl (120 mL) was added, and the mixture was extracted with TBME (2×). The combined org. extracts were dried over MgSO₄, filtered, and the solvents were removed under reduced pressure. Purification of the crude by FC (EtOAc/heptane 1:4) yielded the title compound (1.94 g, 45%). LC-MS: t_{R}=1.06 min; ES+: 326.02.

### (S)-6-[3-(2,6-Dichloro-4-methyl-phenoxy)-pyrrolidin-1-yl]-pyridine-3-carbaldehyde (B5)

BuLi (1.6M in hexane, 5.15 mL, 8.24 mmol) was added to a sol. of compound A3 (2.88 g, 7.16 mmol) in THF (150 mL) at −78° C. The mixture was stirred for 10 min at −78° C., and DMF (0.832 mL, 10.7 mmol) was added. The mixture was stirred for 2.5 h at −78° C., and aq. sat. NH₄Cl (120 mL) was added. The mixture was allowed to warm up to rt, and was extracted with TBME (2×). The combined org. extracts were washed with aq. sat. NH₄Cl, dried over MgSO₄, filtered, and the solvents were removed under reduced pressure. Purification of the crude by FC (EtOAc/heptane 1:4→2:3) yielded the title compound (1.55 g, 61%). LC-MS: t_{R}=0.90 min; ES+: 351.14.

### (E)-2-Cyano-3-{6-[(R)-3-(2,6-dichloro-4-methyl-phenoxy)-pyrrolidin-1-yl]-pyridin-3-yl}-acrylic acid (C1)

Compound B3 (2.97 g, 8.46 mmol) and cyanoacetic acid (719 mg, 8.46 mmol) were dissolved in toluene (85 mL), and piperidine (20 droplets) was added. The mixture was heated to reflux for 4 h, and was allowed to cool to rt. The stirring was stopped, and crystals formed slowly overnight. Crystallization continued at 0° C. The crystals were filtered, and washed with cold heptane. A second crop was obtained from the mother liquors from little CH₂Cl₂/heptane. Drying both crops under high vacuum yielded the title compound (4.06 g, 95%). LC-MS: t_{R}=0.93 min; ES+: 418.09.

### (E)-2-Cyano-3-{6-[2-(2,6-dichloro-4-methyl-phenoxy)-ethoxy]-pyridin-3-yl}-acrylic acid (C2)

A mixture of compound B4 (3.40 g, 10.4 mmol), cyanoacetic acid (887 mg, 10.4 mmol) and piperidine (20 drops) in toluene (96 μL) was heated to reflux for 3 h. The mixture was allowed to cool to rt, while the product precipitated. Filtration, washing with cold heptane and drying this precipitate yielded the title compound (4.01 g, 98%). LC-MS: t_{R}=1.03 min; ES+: 393.07.

### (E)-2-Cyano-3-{6-[(S)-3-(2,6-dichloro-4-methyl-phenoxy)-pyrrolidin-1-yl]-pyridin-3-yl}-acrylic acid (C3)

Compound B5 (1.55 g, 4.40 mmol) and cyanoacetic acid (374 mg, 4.40 mmol) were dissolved in toluene (40 mL), and piperidine (20 droplets) was added. The mixture was heated to reflux overnight, and was allowed to cool to rt. The solvents were removed under reduced pressure. Purification of the crude by FC (CH₂Cl₂/MeOH/AcOH=20:0.5:0.05) yielded the title compound (1.24 g, 67%). LC-MS: t_{R}=0.94 min; ES+: 418.14.

### Mixture of (R)-2-cyano-3-{6-[(R)-3-(2,6-dichloro-4-methyl-phenoxy)-pyrrolidin-1-yl]-pyridin-3-yl}-propionic acid and (S)-2-cyano-3-{6-[(R)-3-(2,6-dichloro-4-methyl-phenoxy)-pyrrolidin-1-yl]-pyridin-3-yl}-propionic acid (D1)

Compound C1 (3.32 g, 7.94 mmol) was dissolved in MeOH (156 mL), and water (78 mL) and NaHCO₃ (567 mg, 5.56 mmol) were added. The mixture was cooled to 0° C., and NaBH₄ (1.80 g, 47.7 mmol) was added in portions over 30 min. The mixture was allowed to warn to rt, and aq. 1M HCl was added till a pH=4 was reached. The solvents were partially removed under reduced pressure, and the residue was extracted with CH₂Cl₂ (3×). The combined org. extracts were dried over MgSO₄, filtered, and the solvents were removed under reduced pressure. Drying under high vacuum yielded the crude title compounds mixture (2.10 g, 63%) that was used without further purification. LC-MS: t_{R}=0.80 min; ES+: 420.10.

### (rac.)-2-Cyano-3-{6-[2-(2,6-dichloro-4-methyl-phenoxy)-ethoxy]-pyridin-3-yl}-propionic acid (D2)

NaHCO₃ (599 mg, 7.13 mmol) and water (100 mL) were added to a sol. of compound C2 (4.01 g, 10.9 mmol) in MeOH (200 mL). The mixture was cooled to 0° C., and NaBH₄ (2.31 g, 61.1 mmol) was added. The mixture was stirred for 75 min at 0° C., and for 4.5 h at rt. Aq. 1M HCl was added to pH 4, and the solvents were partially removed under reduced pressure. The aq. residue was extracted with CH₂Cl₂ (3×). The combined org. extracts were dried over MgSO₄, filtered, and the solvents were removed under reduced pressure. Azeotropic removal of the solvents with toluene yielded the crude title compound (2.60 g, 65%) that was used without further purification. LC-MS: t_{R}=0.97 min; ES+: 395.09.

### Mixture of (R)-2-cyano-3-{6-[(S)-3-(2,6-dichloro-4-methyl-phenoxy)-pyrrolidin-1-yl]-pyridin-3-yl}-propionic acid and (S)-2-cyano-3-{6-[(S)-3-(2,6-dichloro-4-methyl-phenoxy)-pyrrolidin-1-yl]-pyridin-3-yl}-propionic acid (D3)

Compound C3 (1.24 g, 2.95 mmol) was dissolved in MeOH (100 mL), and water (37 mL) and NaHCO₃ (322 mg, 3.84 mmol) were added. The mixture was cooled to 0° C., and NaBH₄ (1.78 g, 45.2 mmol) was added in portions over 4 h. The mixture was allowed to warm to rt, and aq. 1M HCl was added till a pH=4 was reached. The solvents were partially removed under reduced pressure, and the residue was extracted with CH₂Cl₂ (3×). The combined org. extracts were dried over MgSO₄, filtered, and the solvents were removed under reduced pressure. Drying under high vacuum yielded the crude title compounds mixture (1.18 g, 95%) that was used without further purification. LC-MS: t_{R}=0.78 min; ES+: 420.10.

### Mixture of (R)—N-[2-Chloro-5-(3-methoxy-propyl)-benzyl]-2-cyano-N-cyclopropyl-3-{6-[(R)-3-(2,6-dichloro-4-methyl-phenoxy)-pyrrolidin-1-yl]-pyridin-3-yl}-propionamide and (S)—N-[2-Chloro-5-(3-methoxy-propyl)-benzyl]-2-cyano-N-cyclopropyl-3-{6-[(R)-3-(2,6-dichloro-4-methyl-phenoxy)-pyrrolidin-1-yl]-pyridin-3-yl}-propionamide (E1)

A mixture of compounds D1 (700 mg, 1.67 mmol), DMAP (50.8 mg, 0.416 mmol), HOBt (270 mg, 2.00 mmol), DIPEA (1.14 mL, 6.66 mmol) and EDC.HCl (798 mg, 4.16 mmol) in CH₂Cl₂ (35 mL) was stirred for 75 min at rt. [2-Chloro-5-(3-methoxy-propyl)-benzyl]-cyclopropyl-amine (634 mg, 2.50 mmol) was added, and the mixture was stirred for 3 days. The mixture was dissolved with CHCl₃, and the resulting mixture was washed with aq. 1M HCl (2×). The combined aq. layers were extracted back with CHCl₃, and the combined org. extracts were dried over MgSO₄, filtered, and the solvents were removed under reduced pressure. Purification of the crude by FC (MeOH/CH₂Cl₂ 1:99 with 0.1% Et₃N) yielded the title compounds mixture (436 mg, 40%). LC-MS: t_{R}=0.98 min; ES+: 656.96.

### Mixture of (R)—N-[5-chloro-2-(3-methoxy-propyl)-pyridin-4-ylmethyl]-2-cyano-N-cyclopropyl-3-{6-[(R)-3-(2,6-dichloro-4-methyl-phenoxy)-pyrrolidin-1-yl]-pyridin-3-yl}-propionamide and (S)—N-[5-chloro-2-(3-methoxy-propyl)-pyridin-4-ylmethyl]-2-cyano-N-cyclopropyl-3-{6-[(R)-3-(2,6-dichloro-4-methyl-phenoxy)-pyrrolidin-1-yl]-pyridin-3-yl}-propionamide (E2)

A mixture of compounds D1 (700 mg, 1.67 mmol), DMAP (50.8 mg, 0.416 mmol), HOBt (270 mg, 2.00 mmol), DIPEA (1.14 μL, 6.66 mmol) and EDC HCl (798 mg, 4.16 mmol) in CH₂Cl₂ (35 mL) was stirred for 45 min at rt. [5-Chloro-2-(3-methoxy-propyl)-pyridin-4-ylmethyl]-cyclopropyl-amine (637 mg, 2.50 mmol) was added, and the mixture was stirred for 3 days. CHCl₃ was added, and the mixture was washed with aq. 1M HCl (2×). The combined aq. phases were extracted back with CHCl₃. The combined org. extracts were dried over MgSO₄, filtered, and the solvents were removed under reduced pressure. Purification of the residue by FC(CH₂Cl₂/MeOH/Et₃N 20:0.4:0.04) yielded the title compounds mixture (796 mg, 73%). LC-MS: t_{R}=0.91 min; ES+: 657.97.

### (rac.)-N-[2-Chloro-5-(2-methoxy-ethyl)-benzyl]-2-cyano-N-cyclopropyl-3-{6-[2-(2,6-dichloro-4-methyl-phenoxy)-ethoxy]-pyridin-3-yl}-propionamide (E3)

A mixture of compound D2 (600 mg, 1.52 mmol), DMAP (46.4 mg, 0.380 mmol), HOBt (246 mg, 1.82 mmol), DIPEA (1.04 mL, 6.07 mmol) and EDC.HCl (727 mg, 3.08 mmol) in CH₂Cl₂ (26 mL) was stirred for 45 min at rt. [2-Chloro-5-(3-methoxy-ethyl)-benzyl]cyclopropyl-amine (546 mg, 2.28 mmol) was added, and the mixture was stirred for 3 days. CHCl₃ was added, and the mixture was washed with aq. 1M HCl (2×). The combined aq. phases were extracted back with CHCl₃. The combined org. extracts were dried over MgSO₄, filtered, and the solvents were removed under reduced pressure. Purification of the residue by FC(CH₂Cl₂/MeOH/Et₃N 25:0.2:0.05) yielded the title compound (550 mg, 59%). LC-MS: t_{R}=1.19 min; ES+: 616.46.

### (rac.)-N-[2-Chloro-5-(3-methoxy-propyl)-benzyl]-2-cyano-N-cyclopropyl-3-{6-[2-(2,6-dichloro-4-methyl-phenoxy)-ethoxy]-pyridin-3-yl}-propionamide (E4)

A mixture of compound D2 (800 mg, 2.02 mmol), DMAP (61.8 mg, 0.506 mmol), HOBt (328 mg, 2.43 mmol), DIPEA (1.39 mL, 8.10 mmol) and EDC.HCl (970 mg, 5.10 mmol) in CH₂Cl₂ (35 mL) was stirred for 45 min at rt. [2-Chloro-5-(3-methoxy-propyl)-benzyl]-cyclopropyl-amine (771 mg, 3.04 mmol) was added, and the mixture was stirred for 3 days. CHCl₃ was added, and the mixture was washed with aq. 1M HCl (2×). The combined aq. phases were extracted back with CHCl₃. The combined org. extracts were dried over MgSO₄, filtered, and the solvents were removed under reduced pressure. Purification of the residue by FC(CH₂Cl₂/MeOH/Et₃N 25:0.2:0.02) yielded the title compound (860 mg, 67%). LC-MS: t_{R}=1.09 min; ES+: 630.35.

### Mixture of (R)—N-[2-chloro-5-(2-methoxy-ethyl)-benzyl]-2-cyano-N-cyclopropyl-3-{6-[(R)-3-(2,6-dichloro-4-methyl-phenoxy)-pyrrolidin-1-yl]-pyridin-3-yl}-propionamide and (S)—N-[2-chloro-5-(2-methoxy-ethyl)-benzyl]-2-cyano-N-cyclopropyl-3-{6-[(R)-3-(2,6-dichloro-4-methyl-phenoxy)-pyrrolidin-1-yl]-pyridin-3-yl}-propionamide (E5)

A mixture of compounds D1 (350 mg, 0.833 mmol), DMAP (25.4 mg, 0.208 mmol), HOBt (135 mg, 1.00 mmol), DIPEA (0.570 mL, 3.33 mmol) and EDC.HCl (399 mg, 2.08 mmol) in CH₂Cl₂ (18 mL) was stirred for 45 min at rt. [2-Chloro-5-(3-methoxy-ethyl)-benzyl]-cyclopropyl-amine (300 mg, 1.25 mmol) was added, and the mixture was stirred for 3 days. CHCl₃ was added, and the mixture was washed with aq. 1M HCl (2×). The combined aq. phases were extracted back with CHCl₃. The combined org. extracts were dried over MgSO₄, filtered, and the solvents were removed under reduced pressure. Purification of the residue by FC(CH₂Cl₂/MeOH/Et₃N 20:0.3:0.05) yielded the title compounds mixture (461 mg, 86%). LC-MS: t_{R}=0.99 min; ES+: 642.93.

### (rac.)-N-[5-Chloro-2-(3-methoxy-propyl)-pyridin-4-ylmethyl]-2-cyano-N-cyclopropyl-3-{6-[2-(2,6-dichloro-4-methyl-phenoxy)-ethoxy]-pyridin-3-yl}-propionamide (E6)

A mixture of compound D2 (800 mg, 2.02 mmol), DMAP (61.8 mg, 0.506 mmol), HOBt (328 mg, 2.43 mmol), DIPEA (1.39 mL, 8.10 mmol) and EDC.HCl (970 mg, 5.10 mmol) in CH₂Cl₂ (35 mL) was stirred for 45 min at rt. [5-Chloro-2-(3-methoxy-propyl)-pyridin-4-ylmethyl]-cyclopropyl-amine (515 mg, 2.02 mmol) was added, and the mixture was stirred for 3 days. CHCl₃ was added, and the mixture was washed with aq. 1M HCl (2×). The combined aq. phases were extracted back with CHCl₃. The combined org. extracts were dried over MgSO₄, filtered, and the solvents were removed under reduced pressure. Purification of the residue by FC(CH₂Cl₂/MeOH/Et₃N 20:0.3:0.02) yielded the title compound (680 mg, 53%). LC-MS: t_{R}=1.11 min; ES+: 632.97.

### Mixture of (R)—N-[2-Chloro-5-(2-methoxy-ethyl)-benzyl]-2-cyano-N-cyclopropyl-3-{6-[(S)-3-(2,6-dichloro-4-methyl-phenoxy)-pyrrolidin-1-yl]-pyridin-3-yl}-propionamide and (S)—N-[2-Chloro-5-(2-methoxy-ethyl)-benzyl]-2-cyano-N-cyclopropyl-3-{6-[(S)-3-(2,6-dichloro-4-methyl-phenoxy)-pyrrolidin-1-yl]-pyridin-3-yl}-propionamide (E7)

A mixture of compounds D3 (540 mg, 1.29 mmol), DMAP (39.2 mg, 0.321 mmol), HOBt (208 mg, 1.54 mmol), EDC.HCl (616 mg, 3.21 mmol) and DIPEA (1.10 mL, 6.43 mmol) in CH₂Cl₂ (27 mL) was stirred at rt for 45 min. [5-Chloro-2-(3-methoxy-ethyl)-pyridin-4-ylmethyl]-cyclopropyl-amine (532 mg, 1.93 mmol) was added, and the mixture was stirred overnight. CH₂Cl₂ was added, and the mixture was washed with aq. 1M HCl. The org. layer was dried over MgSO₄, filtered, and the solvents were removed under reduced pressure. Purification of the crude by FC(CH₂Cl₂/MeOH/Et₃N=20:0.3:0.05) yielded the title compounds mixture (483 mg, 59%). LC-MS: t_{R}=0.99 min; ES+: 641.16.

### (S)-1-(5-Bromo-pyridin-2-yl)-pyrrolidin-3-ol (F1)

A mixture of 2,5-dibromopyridine (28.6 g, 121 mmol) and (S)-3-hydroxypyrrolidine (10.0 g, 115 mmol) in dry toluene (150 mL) was stirred under reflux for 20 h. The mixture was allowed to cool to rt, and the solvents were removed under reduced pressure. The residue was dissolved with EtOAc, and the resulting mixture was washed with aq. 10% K₂CO₃. The org. layer was dried over MgSO₄, filtered, and the solvents were concentrated under reduced pressure. Purification of the residue by FC(CH₂Cl₂/MeOH 99:1→98:2→97:3→96:4→95:5→94:6→93:7) yielded the title compound (15.39 g, 55%). LC-MS: t_{R}=0.45 min; ES+: 245.11.

### (rac.)-3-Amino-2-{2-[2-(tert-butyl-dimethyl-silanyloxy)-ethoxy]-thiazol-5-ylmethyl}-N-cyclopropyl-N-(2,3-dimethyl-benzyl)-propionamide (L1)

A mixture of (rac.)-2-{2-[2-(tert-butyl-dimethyl-silanyloxy)-ethoxy]-thiazol-5-ylmethyl}-2-cyano-N-cyclopropyl-N-(2,3-dimethyl-benzyl)-acetamide (4.19 g, 8.15 mmol) and CoCl₂ (108 mg, 0.815 mmol) in MeOH (90 mL) was cooled to 0° C., and NaBH₄ (1.28 g, 32.6 mmol) was added in portions. The mixture was stirred at 0° C. for 30 min, and NaBH₄ (642 mg, 16.3 mmol) was added again. The mixture was stirred at 0° C. for 30 min, and NaBH₄ (642 mg, 16.3 mmol) was added again. The mixture was stirred at 0° C. for 2 h, and NaBH₄ (642 mg, 16.3 mmol) and MeOH (30 mL) were added. The mixture was stirred for 2 h at 0° C., and was filtered over celite. The filtrate was evaporated under reduced pressure, and the residue was partitioned between CH₂Cl₂ and aq. 1M NaOH. The org. layer was dried over Na₂SO₄, filtered, and the solvents were removed under reduced pressure. Drying the residue under high vacuum yielded the crude title compound (4.18 g, 99%) that was used without further purification. LC-MS: t_{R}=0.95 min; ES+: 518.46.

### Mixture of (R)-2-aminomethyl-3-{2-[(S)-3-(tert-butyl-dimethyl-silanyloxy)-pyrrolidin-1-yl]-thiazol-5-yl}-N-cyclopropyl-N-(2,3-dichloro-benzyl)-propionamide and (S)-2-aminomethyl-3-{2-[(S)-3-(tert-butyl-dimethyl-silanyloxy)-pyrrolidin-1-yl]-thiazol-5-yl}-N-cyclopropyl-N-(2,3-dichloro-benzyl)-propionamide (L2)

A mixture of (R)-3-{2-[(S)-3-(tert-butyl-dimethyl-silanyloxy)-pyrrolidin-1-yl]-thiazol-5-yl}-2-cyano-N-cyclopropyl-N-(2,3-dichloro-benzyl)-propionamide and (S)-3-{2-[(S)-3-(tert-butyl-dimethyl-silanyloxy)-pyrrolidin-1-yl]-thiazol-5-yl}-2-cyano-N-cyclopropyl-N-(2,3-dichloro-benzyl)-propionamide (3.66 g, 6.31 mmol) and CoCl₂ (83.6 mg, 0.631 mmol) in MeOH (50 mL) was cooled to 0° C., and NaBH₄ (746 mg, 18.9 mmol) was added in portions. The mixture was stirred for a total time of 6 h at 0° C., whereas NaBH₄ (498 mg, 12.6 mmol) was added every 2 h. After the last addition, the mixture was stirred for 1 h at rt, and was filtered over celite. The filtrate was reduced under reduced pressure, and partitioned between CH₂Cl₂ and aq. 1M NaOH. The org. layer was dried over Na₂SO₄, filtered, and the solvents were removed under reduced pressure. Purification of the crude by FC (MeOH/CH₂Cl₂ 1:15) yielded the title compounds mixture (1.71 g, 46%). LC-MS: t_{R}=0.79 min; ES+: 583.31.

### (rac.)-{3-{2-[2-(tert-Butyl-dimethyl-silanyloxy)-ethoxy]-thiazol-5-yl}-2-[cyclopropyl-(2,3-dimethyl-benzyl)-carbamoyl]-propyl}-carbamic acid tert-butyl ester (M1)

Boc₂O (2.70 g, 12.1 mmol) was added to a sol. of compound L1 (4.18 g, 8.07 mmol) and DIPEA (2.82 mL, 16.1 mmol) in CH₂Cl₂ (150 mL) at 0° C. The mixture was stirred for 3 days, while warming up to rt. The mixture was cooled to 0° C., and was washed with aq. 1M HCl (2×) and aq. sat. NaHCO₃ (1×). The combined aq. phases were extracted back with CH₂Cl₂ (1×). The combined org. extracts were dried over MgSO₄, filtered, and the solvents were removed under reduced pressure. Purification of the crude by FC (MeOH/CH₂Cl₂ 1:66) yielded the title compound (2.14 g, 43%). LC-MS: t_{R}=1.20 min; ES+: 618.56.

### (rac.)-{2-[Cyclopropyl-(2,3-dimethyl-benzyl)-carbamoyl]-3-[2-(2-hydroxy-ethoxy)-thiazol-5-yl]-propyl}-carbamic acid tert-butyl ester (M2)

TBAF (1M in THF, 6.90 mL; 6.90 mmol) was added to a sol. of compound M1 (2.14 g, 3.46 mmol) in THF (50 mL) at 0° C. The mixture was stirred for 60 min at 0° C., and aq. sat. NH₄Cl was added. The mixture was extracted with Et₂O (2×). The combined org. extracts were dried over MgSO₄, filtered, and the solvents were removed under reduced pressure. Purification of the crude by FC (MeOH/CH₂Cl₂ 1:45→1:40→1:35) yielded the title compound (1.18 g, 68%). LC-MS: t_{R}=0.95 min; ES+: 504.44.

### Mixture of (R)-{3-{2-[(S)-3-(tert-Butyl-dimethyl-silanyloxy)-pyrrolidin-1-yl]-thiazol-5-yl}-2-[cyclopropyl-(2,3-dichloro-benzyl)-carbamoyl]-propyl}-carbamic acid tert-butyl ester and (S)-{3-{2-[(S)-3-(tert-Butyl-dimethyl-silanyloxy)-pyrrolidin-1-yl]-thiazol-5-yl}-2-[cyclopropyl-(2,3-dichloro-benzyl)-carbamoyl]-propyl}-carbamic acid tert-butyl ester (M3)

Boc₂O (979 mg, 4.39 mmol) was added to a sol. of compounds L2 (1.71 g, 2.93 mmol) and DIPEA (1.02 mL, 5.86 mmol) in CH₂Cl₂ (50 mL) at 0° C. The mixture was stirred overnight, while warming up to rt. Ice was added, and the mixture was washed with cold aq. 1M HCl (2×) and aq. sat. NaHCO₃ (1×). The combined aq. layers were extracted back with CH₂Cl₂ (1×). The combined org. extracts were dried over MgSO₄, filtered, and the solvents were removed under reduced pressure. Purification of the crude by FC (MeOH/CH₂Cl₂ 1:19) yielded the title compounds mixture (1.85 g, 93%). LC-MS: t_{R}=1.03 min; ES+: 683.37.

### Mixture of (R)-{2-[cyclopropyl-(2,3-dichloro-benzyl)-carbamoyl]-3-[2-((S)-3-hydroxy-pyrrolidin-1-yl)-thiazol-5-yl]-propyl}-carbamic acid tert-butyl ester and (S)-{2-[cyclopropyl-(2,3-dichloro-benzyl)-carbamoyl]-3-[2-((S)-3-hydroxy-pyrrolidin-1-yl) thiazol-5-yl]-propyl}-carbamic acid tert-butyl ester (M4)

TBAF (1M in THF, 5.4 mL; 5.4 mmol) was added to a sol. of compounds M3 (1.85 g, 2.71 mmol) in THF (40 mL) at 0° C. The mixture was stirred for 60 min at 0° C., and aq. sat. NH₄Cl was added. The mixture was extracted with Et₂O (2×). The combined org. extracts were dried over MgSO₄, filtered, and the solvents were removed under reduced pressure. Purification of the crude by FC (MeOH/CH₂Cl₂ 1:45→1:40→1:35) yielded the title compounds mixture (1.48 g, 96%). LC-MS: t_{R}=0.85 min; ES+: 569.37.

### (rac.)-(2-[Cyclopropyl-(2,3-dimethyl-benzyl)-carbamoyl]-3-{2-[2-(2,6-dichloro-4-methyl-phenoxy)-ethoxy]-thiazol-5-yl}-propyl)-carbamic acid tert-butyl ester (N1)

According to General procedure A, from compound M2 and 2,6-dichloro-p-cresol. LC-MS: t_{R}=1.20 min; ES+: 662.36.

### (rac.)-{3-{2-[2-(2-Chloro-3,6-difluoro-phenoxy)-ethoxy]-thiazol-5-yl}-2-[cyclopropyl-(2,3-dimethyl-benzyl)-carbamoyl]-propyl}-carbamic acid tert-butyl ester (N2)

According to General procedure A, from compound M2 and 2-chloro-3,6-difluorophenol. LC-MS: t_{R}=1.13 min; ES+: 650.41.

### (rac.)-(2-[Cyclopropyl-(2,3-dimethyl-benzyl)-carbamoyl]-3-{2-[2-(2,6-dichloro-phenoxy)-ethoxy]-thiazol-5-yl}-propyl)-carbamic acid tert-butyl ester (N3)

According to General procedure A, from compound M2 and 2,6-dichlorophenol. LC-MS: t_{R}=1.17 min; ES+: 648.33.

### (rac.)-(2-[Cyclopropyl-(2,3-dimethyl-benzyl)-carbamoyl]-3-{2-[2-(2,6-dichloro-3,4-dimethyl-phenoxy)-ethoxy]-thiazol-5-yl}-propyl)-carbamic acid tert-butyl ester (N4)

According to General procedure A, from compound M2 and 2,6-dichloro-3,4-dimethylphenol. LC-MS: t_{R}=1.22 min; ES+: 676.39.

### (rac.)-{3-{2-[2-(2-Chloro-6-fluoro-3-methyl-phenoxy)-ethoxy]-thiazol-5-yl}-2-[cyclopropyl-(2,3-dimethyl-benzyl)-carbamoyl]-propyl}-carbamic acid tert-butyl ester (N5)

According to General procedure A, from compound M2 and 2-chloro-6-fluoro-3-methylphenol. LC-MS: t_{R}=1.18 min; ES+: 646.40.

### Mixture of (rac.)-(R*)-{3-[2-(2-{(R*)-4-[1-(tert-butyl-dimethyl-silanyloxy)-ethyl]-2,6-dichloro-phenoxy}-ethoxy)-thiazol-5-yl]-2-[cyclopropyl-(2,3-dimethyl-benzyl)-carbamoyl]-propyl}-carbamic acid tert-butyl ester and (rac.)-(R*)-{3-[2-(2-{(S*)-4-[1-(tert-butyl-dimethyl-silanyloxy)-ethyl]-2,6-dichloro-phenoxy}-ethoxy)thiazol-5-yl-2-[cyclopropyl-(2,3-dimethyl-benzyl)-carbamoyl]-propyl}-carbamic acid tert-butyl ester (N6)

According to General procedure A, from compound M2 and (rac.)-4-[1-(tert-butyldimethylsilanyloxy)ethyl]-2,6-dichlorophenol. LC-MS: t_{R}=1.31 min; ES+: 808.47.

### (rac.)-{3-{2-[2-(3-Chloro-2,6-difluoro-phenoxy)-ethoxy]-thiazol-5-yl}-2-[cyclopropyl-(2,3-dimethyl-benzyl)-carbamoyl]-propyl}-carbamic acid tert-butyl ester (N7)

According to General procedure A, from compound M2 and 3-chloro-2,6-difluorophenol. LC-MS: t_{R}=1.16 min; ES+: 650.39.

### (rac.)-(2-[Cyclopropyl-(2,3-dimethyl-benzyl)-carbamoyl]-3-{2-[2-(2,6-dichloro-4-fluoro-phenoxy)-ethoxy]-thiazol-5-yl}-propyl)-carbamic acid tert-butyl ester (N8)

According to General procedure A, from compound M2 and 2,6-dichloro-4-fluorophenol. LC-MS: t_{R}=1.18 min; ES+: 666.38.

### Mixture of ((R)-2-[cyclopropyl-(2,3-dichloro-benzyl)-carbamoyl]-3-{2-[(R)-3-(2,6-dichloro-4-methyl-phenoxy)-pyrrolidin-1-yl]-thiazol-5-yl}-propyl)-carbamic acid tert-butyl ester and ((S)-2-[cyclopropyl-(2,3-dichloro-benzyl)-carbamoyl]-3-{2-[(R)-3-(2,6-dichloro-4-methyl-phenoxy)-pyrrolidin-1-yl]-thiazol-5-yl}-propyl)-carbamic acid tert-butyl ester (N9)

According to General procedure A, from compounds M4 and 2,6-dichloro-p-cresol. LC-MS: t_{R}=1.04 min; ES+: 729.18.

### Mixture of ((R)-2-[cyclopropyl-(2,3-dichloro-benzyl)-carbamoyl]-3-{2-[(R)-3-(2,6-dichloro-phenoxy)-pyrrolidin-1-yl]-thiazol-5-yl}-propyl)-carbamic acid tert-butyl ester and ((S)-2-[cyclopropyl-(2,3-dichloro-benzyl)-carbamoyl]-3-{2-[(R)-3-(2,6-dichloro-phenoxy)-pyrrolidin-1-yl]-thiazol-5-yl}-propyl)-carbamic acid tert-butyl ester (N10)

According to General procedure A, from compounds M4 and 2,6-dichlorophenol. LC-MS: t_{R}=1.02 min; ES+: 715.28.

### Mixture of ((R)-2-[cyclopropyl-(2,3-dichloro-benzyl)-carbamoyl]-3-{2-[(R)-3-(2,6-dichloro-3,4-dimethyl-phenoxy)-pyrrolidin-1-yl]-thiazol-5-yl}-propyl)-carbamic acid tert-butyl ester and ((S)-2-[cyclopropyl-(2,3-dichloro-benzyl)-carbamoyl]-3-{2-[(R)-3-(2,6-dichloro-3,4-dimethyl-phenoxy)-pyrrolidin-1-yl]-thiazol-5-yl}-propyl)-carbamic acid tert-butyl ester (N11)

According to General procedure A, from compounds M4 and 2,6-dichloro-3,4-dimethylphenol. LC-MS: t_{R}=1.06 min; ES+: 741.37.

### Mixture of {(R)-3-{2-[(R)-3-(2-chloro-6-fluoro-3-methyl-phenoxy)-pyrrolidin-1-yl]-thiazol-5-yl}-2-[cyclopropyl-(2,3-dichloro-benzyl)-carbamoyl]-propyl}-carbamic acid tert-butyl ester and {(S)-3-{2-[(R)-3-(2-chloro-6-fluoro-3-methyl-phenoxy)-pyrrolidin-1-yl]-thiazol-5-yl}-2-[cyclopropyl-(2,3-dichloro-benzyl)-carbamoyl]-propyl}-carbamic acid tert-butyl ester (N12)

According to General procedure A, from compounds M4 and 2-chloro-6-fluoro-3-methylphenol. LC-MS: t_{R}=1.02 min; ES+: 713.32.

### Mixture of {(R)-3-[2-((R)-3-{(R)-4-[1-(tert-butyl-dimethyl-silanyloxy)-ethyl]-2,6-dichloro-phenoxy}-pyrrolidin-1-yl)-thiazol-5-yl]-2-[cyclopropyl-(2,3-dichloro-benzyl)-carbamoyl]-propyl}-carbamic acid tert-butyl ester, {(R)-3-[2-((R)-3-{(S)-4-[1-(tert-butyl-dimethyl-silanyloxy)-ethyl]-2,6-dichloro-phenoxy}-pyrrolidin-1-yl)-thiazol-5-yl]-2-[cyclopropyl-(2,3-dichloro-benzyl)-carbamoyl]-propyl}-carbamic acid tert-butyl ester, {(S)-3-[2-((R)-3-{(R)-4-[1-(tert-butyl-dimethyl-silanyloxy)-ethyl]-2,6-dichloro-phenoxy}-pyrrolidin-1-yl)-thiazol-5-yl]-2-[cyclopropyl-(2,3-dichloro-benzyl)-carbamoyl]-propyl}-carbamic acid tert-butyl ester, and {(S)-3-[2-((R)-3-{(S)-4-[1-(tert-butyl-dimethyl-silanyloxy)-ethyl]-2,6-dichloro-phenoxy}-pyrrolidin-1-yl)-thiazol-5-yl]-2-[cyclopropyl-(2,3-dichloro-benzyl)-carbamoyl]-propyl}-carbamic acid tert-butyl ester (N13)

According to General procedure A, from compounds M4 and (rac.)-4-[1-(tert-butyldimethylsilanyloxy)ethyl]-2,6-dichlorophenol. LC-MS: t_{R}=1.15 min; ES+: 873.44.

### Mixture of {(R)-3-{2-[(R)-3-(3-chloro-2,6-difluoro-phenoxy)-pyrrolidin-1-yl]-thiazol-5-yl}-2-[cyclopropyl-(2,3-dichloro-benzyl)-carbamoyl]-propyl}-carbamic acid tert-butyl ester and {(S)-3-{2-[(R)-3-(3-chloro-2,6-difluoro-phenoxy)-pyrrolidin-1-yl]-thiazol-5-yl}-2-[cyclopropyl-(2,3-dichloro-benzyl)-carbamoyl]-propyl}-carbamic acid tert-butyl ester (N14)

According to General procedure A, from compounds M4 and 3-chloro-2,6-difluorophenol. LC-MS: t_{R}=1.01 min; ES+: 717.35.

### Mixture of ((R)-2-[cyclopropyl-(2,3-dichloro-benzyl)-carbamoyl]-3-{2-[(R)-3-(2,6-dichloro-4-fluoro-phenoxy)-pyrrolidin-1-yl]-thiazol-5-yl}-propyl)-carbamic acid tert-butyl ester and ((S)-2-[cyclopropyl-(2,3-dichloro-benzyl)-carbamoyl]-3-{2-[(R)-3-(2,6-dichloro-4-fluoro-phenoxy)-pyrrolidin-1-yl]-thiazol-5-yl}-propyl)-carbamic acid tert-butyl ester (N15)

According to General procedure A, from compounds M4 and 2,6-dichloro-4-fluorophenol. LC-MS: t_{R}=1.02 min; ES+: 733.32.

### EXAMPLES

### Example 1

### (rac.)-3-Amino-N-cyclopropyl-2-[2-{2-(2,6-dichloro-4-methyl-phenoxy)-ethoxy]-thiazol-5-ylmethyl}-N-(2,3-dimethyl-benzyl)-propionamide

According to General procedure B, from compound N1. LC-MS: t_{R}=0.94 min; ES+: 562.33.

### Example 2

### (rac.)-3-Amino-2-{2-[2-(2-chloro-3,6-difluoro-phenoxy)-ethoxy]-thiazol-5-ylmethyl}-N-cyclopropyl-N-(2,3-dimethyl-benzyl)-propionamide

According to General procedure B, from compound N2. LC-MS: t_{R}=0.89 min; ES+: 550.30.

### Example 3

### (rac.)-3-Amino-N-cyclopropyl-2-{2-[2-(2,6-dichloro-phenoxy)-ethoxy]-thiazol-5-ylmethyl}-N-(2,3-dimethyl-benzyl)-propionamide

According to General procedure B, from compound N3. LC-MS: t_{R}=0.92 min; ES+: 550.26.

### Example 4

### (rac.)-3-Amino-N-cyclopropyl-2-{2-[2-(2,6-dichloro-3,4-dimethyl-phenoxy)-ethoxy]-thiazol-5-ylmethyl}-N-(2,3-dimethyl-benzyl)-propionamide

According to General procedure B, from compound N4. LC-MS: t_{R}=0.97 min; ES+: 576.32.

### Example 5

### (rac.)-3-Amino-2-{2-[2-(2-chloro-6-fluoro-3-methyl-phenoxy)-ethoxy]-thiazol-5-ylmethyl}-N-cyclopropyl-N-(2,3-dimethyl-benzyl)-propionamide

According to General procedure B, from compound N5. LC-MS: t_{R}=0.92 min; ES+: 546.26.

### Example 6

### (rac.)-(R*)-3-Amino-N-cyclopropyl-2-(2-{2-[2,6-dichloro-4-((R*)-1-hydroxy-ethyl)-phenoxy]-ethoxy}-thiazol-5-ylmethyl)-N-(2,3-dimethyl-benzyl)-propionamide and (rac.)-(R*)-3-amino-N-cyclopropyl-2-(2-{2-[2,6-dichloro-4-((S*)-1-hydroxy-ethyl)-phenoxy]-ethoxy}-thiazol-5-ylmethyl)-N-(2,3-dimethyl-benzyl)-propionamide

According to General procedure B, from compound N6. LC-MS: t_{R}=0.87 min; ES+: 592.31.

### Example 7

### (rac.)-3-Amino-2-{2-[2-(3-chloro-2,6-difluoro-phenoxy)-ethoxy]-thiazol-5-ylmethyl}-N-cyclopropyl-N-(2,3-dimethyl-benzyl)-propionamide

According to General procedure B, from compound N7. LC-MS: t_{R}=0.92 min; ES+: 550.25.

### Example 8

### (rac.)-3-Amino-N-cyclopropyl-2-{2-[2-(2,6-dichloro-4-fluoro-phenoxy)-ethoxy]-thiazol-5-ylmethyl}-N-(2,3-dimethyl-benzyl)-propionamide

According to General procedure B, from compound N8. LC-MS: t_{R}=0.93 min; ES+: 566.36.

### Example 9

### Mixture of (R)-2-aminomethyl-N-cyclopropyl-N-(2,3-dichloro-benzyl)-3-{2-[(R)-3-(2,6-dichloro-4-methyl-phenoxy)-pyrrolidin-1-yl]-thiazol-5-yl}-propionamide and (S)-2-aminomethyl-N-cyclopropyl-N-(2,3-dichloro-benzyl)-3-{2-[(R)-3-(2,6-dichloro-4-methyl-phenoxy)-pyrrolidin-1-yl]-thiazol-5-yl}-propionamide

According to General procedure B, from compounds N9. LC-MS: t_{R}=0.84 min; ES+: 629.18.

### Example 10

### Mixture of (R)-2-aminomethyl-N-cyclopropyl-N-(2,3-dichloro-benzyl)-3-{2-[(R)-3-(2,6-dichloro-phenoxy)-pyrrolidin-1-yl]-thiazol-5-yl}-propionamide and (S)-2-aminomethyl-N-cyclopropyl-N-(2,3-dichloro-benzyl)-3-{2-[(R)-3-(2,6-dichloro-phenoxy)-pyrrolidin-1-yl]-thiazol-5-yl}-propionamide

According to General procedure B, from compounds N10. LC-MS: t_{R}=0.82 min; ES+: 613.19.

### Example 11

### Mixture of (R)-2-aminomethyl-N-cyclopropyl-N-(2,3-dichloro-benzyl)-3-{2-[(R)-3-(2,6-dichloro-3,4-dimethyl-phenoxy)-pyrrolidin-1-yl]-thiazol-5-yl}-propionamide and (S)-2-aminomethyl-N-cyclopropyl-N-(2,3-dichloro-benzyl)-3-{2-[(R)-3-(2,6-dichloro-3,4-dimethyl-phenoxy)-pyrrolidin-1-yl]-thiazol-5-yl}-propionamide

According to General procedure B, from compounds N11. LC-MS: t_{R}=0.86 min; ES+: 643.09.

### Example 12

### Mixture of (R)-2-aminomethyl-3-{2-[(R)-3-(2-chloro-6-fluoro-3-methyl-phenoxy)-pyrrolidin-1-yl]-thiazol-5-yl}-N-cyclopropyl-N-(2,3-dichloro-benzyl)-propionamide and (S)-2-aminomethyl-3-{2-[(R)-3-(2-chloro-6-fluoro-3-methyl-phenoxy)-pyrrolidin-1-yl]-thiazol-5-yl}-N-cyclopropyl-N-(2,3-dichloro-benzyl)-propionamide

According to General procedure B, from compounds N12. LC-MS: t_{R}=0.82 min; ES+: 611.21.

### Example 13

### Mixture of (R)-2-aminomethyl-N-cyclopropyl-N-(2,3-dichloro-benzyl)-3-(2-{(R)-3-[(R)-2,6-dichloro-4-(1-hydroxy-ethyl)-phenoxy]-pyrrolidin-1-yl}-thiazol-5-yl)-propionamide, (S)-2-aminomethyl-N-cyclopropyl-N-(2,3-dichloro-benzyl)-3-(2-{(R)-3-[(R)-2,6-dichloro-4-(1-hydroxy-ethyl)-phenoxy]-pyrrolidin-1-yl}-thiazol-5-yl)-propionamide, (R)-2-aminomethyl-N-cyclopropyl-N-(2,3-dichloro-benzyl)-3-(2-{(R)-3-[(S)-2,6-dichloro-4-(1-hydroxy-ethyl)-phenoxy]-pyrrolidin-1-yl}-thiazol-5-yl)-propionamide, and (S)-2-aminomethyl-N-cyclopropyl-N-(2,3-dichloro-benzyl)-3-(2-{(R)-3-[(S)-2,6-dichloro-4-(1-hydroxy-ethyl)-phenoxy]-pyrrolidin-1-yl}-thiazol-5-yl)-propionamide

According to General procedure B, from compounds N13. LC-MS: t_{R}=0.78 min; ES+: 659.23.

### Example 14

### Mixture of (R)-2-aminomethyl-3-{2-[(R)-3-(3-chloro-2,6-difluoro-phenoxy)-pyrrolidin-1-yl]-thiazol-5-yl}-N-cyclopropyl-N-(2,3-dichloro-benzyl)-propionamide and (S)-2-aminomethyl-3-{2-[(R)-3-(3-chloro-2,6-difluoro-phenoxy)-pyrrolidin-1-yl]-thiazol-5-yl}-N-cyclopropyl-N-(2,3-dichloro-benzyl)-propionamide

According to General procedure B, from compounds N14. LC-MS: t_{R}=0.82 min; ES+: 617.24.

### Example 15

### Mixture of (R)-2-aminomethyl-N-cyclopropyl-N-(2,3-dichloro-benzyl)-3-{2-[(R)-3-(2,6-dichloro-4-fluoro-phenoxy)-pyrrolidin-1-yl]-thiazol-5-yl}-propionamide and (S)-2-aminomethyl-N-cyclopropyl-N-(2,3-dichloro-benzyl)-3-{2-[(R)-3-(2,6-dichloro-4-fluoro-phenoxy)-pyrrolidin-1-yl]-thiazol-5-yl}-propionamide

According to General procedure B, from compounds N15. LC-MS: t_{R}=0.83 min; ES+: 633.22.

### Example 16

### (R)-2-Aminomethyl-N-[2-chloro-5-(3-methoxy-propyl)-benzyl]-N-cyclopropyl-3-{6-[(R)-3-(2,6-dichloro-4-methyl-phenoxy)-pyrrolidin-1-yl]-pyridin-3-yl}-propionamide

CoCl₂ (17.3 mg, 0.133 mmol) was added to a sot. of compounds E1 (436 mg, 0.665 mmol) in MeOH (8.00 mL) at 0° C. NaBH₄ (101 mg, 2.66 mmol) was added in portions. The mixture was stirred for 90 min, and CH₂Cl₂ and aq. 1M NaOH were added. The mixture was filtered through celite, and the phases were separated. The org. phase was washed with aq. 1M NaOH, water, and brine. The org. layer was dried over MgSO₄, filtered, and the solvents were removed under reduced pressure. Purification of the crude by FC (MeOH/CH₂Cl₂ 1:20 with 0.1% Et₃N) yielded the title compound mixed with its corresponding (S,R)-diastereoisomer (241 mg, 55%). Separation of this mixture by HPLC using a chiral stationary phase (Regis Whelk column, 10 μm, 50 m×250 mm, 120 mL/min, gradient from EtOH/hexane 25:75 with 0.15% Et₃N to EtOH/hexane 70:30 with 0.15% Et₃N over 30 min) yielded the title compound. LC-MS: t_{R}=0.98 min; ES+: 656.22. Chiral, preparative Regis Whelk 01 column: t_{R}=25.9 min.

### Example 17

### (R)-2-Aminomethyl-N-[5-chloro-2-(3-methoxy-propyl)-pyridin-4-ylmethyl]-N-cyclopropyl-3-{6-[(R)-3-(2,6-dichloro-4-methyl-phenoxy)-pyrrolidin-1-yl]-pyridin-3-yl}-propionamide

CoCl₂ (41.9 mg, 0.323 mmol) was added to a sol. of compounds E2 (796 mg, 1.21 mmol) in MeOH (13 mL). NaBH₄ (183 mg, 4.85 mmol) was added in portions, and the mixture was stirred for 4 h at 0° C. The mixture was diluted with CH₂Cl₂ and aq. 1M NaOH. The mixture was filtered over celite, and the phases were separated. The org. layer was washed with aq. 1M NaOH and brine, was dried over MgSO₄, filtered, and the solvents were removed under reduced pressure. Purification of the residue by FC(CH₂Cl₂/MeOH/Et₃N 25:1.0:0.1) yielded the title compound mixed with its corresponding (S,R)-diastereoisomer. Separation of this mixture by HPLC using a chiral stationary phase (Regis Whelk column, 10 μm, 50 m×250 mm, 120 mL/min, gradient from EtOH/hexane 25:75 with 0.15% Et₃N to EtOH/hexane 70:30 with 0.15% Et₃N over 30 min) yielded the title compound. LC-MS: t_{R}=0.78 min; ES+: 660.21. Chiral, preparative Regis Whelk 01 column: t_{R}=27.9 min.

### Example 18

### (R)-2-Aminomethyl-N-[2-chloro-5-(2-methoxy-ethyl)-benzyl]-N-cyclopropyl-3-{6-[2-(2,6-dichloro-4-methyl-phenoxy)-ethoxy]-pyridin-3-yl}-propionamide

CoCl₂ (23.1 mg, 0.178 mmol) was added to a sol. of compound E3 (550 mg, 0.891 mmol) in MeOH (11 mL) at 0° C. NaBH₄ (135 mg, 3.57 mmol) was added in portions, and the mixture was stirred for 30 min. CH₂Cl₂ was added, and the mixture was washed with aq. 1M NaOH. The org. layer was dried over MgSO₄, filtered, and the solvents were removed under reduced pressure. Purification of the crude by FC(CH₂Cl₂/MeOH/Et₃N=25:1:0.1) yielded the racemic title compound (236 mg, 43%). Separation of this mixture by HPLC using a chiral stationary phase (Regis Whelk column, 10 μm, 50 m×250 mm, 120 mL/min, gradient from EtOH/hexane 25:75 with 0.15% Et₃N to EtOH/hexane 70:30 with 0.15% Et₃N over 30 min) yielded the title compound (73 mg, 37%). LC-MS: t_{R}=0.93 min; ES+: 619.94. Chiral, preparative Regis Whelk 01 column: t_{R}=16.6 min.

### Example 19

### (R)-2-Aminomethyl-N-[2-chloro-5-(3-methoxy-propyl)-benzyl]-N-cyclopropyl-3-{6-[2-(2,6-dichloro-4-methyl-phenoxy)-ethoxy]-pyridin-3-yl}-propionamide

CoCl₂ (35.4 mg, 0.273 mmol) was added to a sol. of compound E4 (860 mg, 1.36 mmol) in MeOH (15 mL) at 0° C. NaBH₄ (306 mg, 5.45 mmol) was added in portions, and the mixture was stirred for 30 min. CH₂Cl₂ was added, and the mixture was washed with aq. 1M NaOH. The org. layer was dried over MgSO₄, filtered, and the solvents were removed under reduced pressure. Purification of the crude by FC(CH₂Cl₂/MeOH/Et₃N=25:1:0.1) yielded the racemic title compound (453 mg, 52%). Separation of this mixture by HPLC using a chiral stationary phase (Regis Whelk column, 10 μm, 50 m×250 mm, 120 mL/min, isocratic conditions EtOH/hexane 40:60 with 0.1% Et₃N) yielded the title compound (100 mg, 23%). LC-MS: t_{R}=0.96 min; ES+: 636.43. Chiral, preparative Regis Whelk 01 column: t_{R}=14.3 min.

### Example 20

### (R)-2-Aminomethyl-N-[2-chloro-5-(2-methoxy-ethyl)-benzyl]-N-cyclopropyl-3-{6-[(R)-3-(2,6-dichloro-4-methyl-phenoxy)-pyrrolidin-1-yl]-pyridin-3-yl}-propionamide

CoCl₂ (18.7 mg, 0.144 mmol) was added to a sol. of compounds E5 (461 mg, 0.718 mmol) in MeOH (8.6 mL) at 0° C. NaBH₄ (109 mg, 2.87 mmol) was added in portions, and the mixture was stirred for 30 min. CH₂Cl₂ was added, and the mixture was washed with aq. 1M NaOH. The org. layer was dried over MgSO₄, filtered, and the solvents were removed under reduced pressure. Purification of the crude by FC(CH₂Cl₂/MeOH/Et₃N=25:1:0.1) yielded the title compound mixed with its diastereoisomer from compounds E5 (229 mg, 50%). Separation of this mixture by HPLC using a chiral stationary phase (Regis Whelk column, 10 μm, 50 m×250 mm, 120 mL/min, gradient from EtOH/hexane 25:75 with 0.15% Et₃N to EtOH/hexane 70:30 with 0.15% Et₃N over 30 min) yielded the title compound (62 mg, 27%). LC-MS: t_{R}=0.96 min; ES+: 636.43. Chiral, preparative Regis Whelk 01 column: t_{R}=25.9 min.

### Example 21

### (R)-2-Aminomethyl-N-[5-chloro-2-(3-methoxy-propyl)-pyridin-4-ylmethyl]-N-cyclopropyl-3-{6-[2-(2,6-dichloro-4-methyl-phenoxy)-ethoxy]-pyridin-3-yl}-propionamide

CoCl₂ (20.0 mg, 0.154 mmol) was added to a sol. of compound E6 (470 mg, 0.744 mmol) in MeOH (8.0 mL) at 0° C. NaBH₄ (200 mg, 3.17 mmol) was added in portions, and the mixture was stirred for 30 min. CH₂Cl₂ was added, and the mixture was washed with aq. 1M NaOH. The org. layer was dried over MgSO₄, filtered, and the solvents were removed under reduced pressure. Purification of the crude by FC(CH₂Cl₂/MeOH/Et₃N=20:1:0.1) yielded the racemic title compound (275 mg, 58%). Separation of this mixture by HPLC using a chiral stationary phase (Regis Whelk column, 10 μm, 50 m×250 mm, 120 mL/min, isocratic conditions EtOH/hexane 40:60 with 0.1% Et₃N) yielded the title compound (93 mg, 28%). LC-MS: t_{R}=0.87 min; ES+: 636.99. Chiral, preparative Regis Whelk 01 column: t_{R}=17.0 min.

### Example 22

### (R)-2-Aminomethyl-N-[2-chloro-5-(2-methoxy-ethyl)-benzyl]-N-cyclopropyl-3-{6-[(S)-3-(2,6-dichloro-4-methyl-phenoxy)-pyrrolidin-1-yl]-pyridin-3-yl}-propionamide

CoCl₂ (19.6 mg, 0.151 mmol) was added to a sol. of compounds E7 (483 mg, 0.753 mmol) in MeOH (9.3 mL) at 0° C. NaBH₄ (114 mg, 3.02 mmol) was added in portions, and the mixture was stirred for 30 min. CH₂Cl₂ was added, and the mixture was washed with aq. 1M NaOH. The org. layer was dried over MgSO₄, filtered, and the solvents were removed under reduced pressure. Purification of the crude by FC(CH₂Cl₂/MeOH/Et₃N=20:1:0.1) yielded the title compound together with its (S,S)-diastereoisomer (300 mg, 62%). Separation of this mixture by HPLC using a chiral stationary phase (Regis Whelk column, 10 μm, 50 m×250 mm, 120 mL/min, isocratic conditions EtOH/hexane 40:60 with 0.1% Et₃N) yielded the title compound (75 mg, 25%). LC-MS: t_{R}=0.82 min; ES+: 645.20. Chiral, preparative Regis Whelk 01 column: t_{R}=29.1 min.

### Biological Assays

### 1. Enzyme Immuno Assay (EIA) to Estimate AngI Accumulation and Renin Inhibition

### 1.1 Preparation of AngI-BSA Conjugate

1.3 mg (1 μmol) of AngI [1-10 (Bachem, H-1680)] and 17 mg (0.26 μmol) of BSA (Fluka, 05475) were dissolved in 4 mL of 0.1M phosphate buffer, pH 7.4, after which 2 mL of a 1:100 dilution of glutaraldehyde in H₂O (Sigma G-5882) was added dropwise. The mixture was incubated overnight at 4° C., then dialyzed against 2 liters of 0.9% NaCl, twice for 4 h at rt, followed by dialysis against 2 liters of PBS 1× overnight at rt. The solution was then filtered with a Syringe filter, 0.45 μm (Nalgene, Cat. No. 194-2545). The conjugate can be stored in polypropylene tubes in 0.05% sodium azide at 4° C. for at least 12 months.

### 1.2 Preparation of BSA-AngI Coated MTP

Microtiter plates (MPT384, MaxiSorp™, Nunc) were incubated overnight at 4° C. with 80 μl of AngI (1-10)/BSA conjugate, diluted 1:100'000 in PBS 1× in a teflon beaker (exact dilution dependent on batch of conjugate), emptied, filled with 90 μl of blocking solution [0.5% BSA (Sigma A-2153) in PBS 1×, 0.02% NaN₃], and incubated for at least 2 h at rt, or overnight at 4° C. 96 well MTP (MaxiSorp™, Nunc) were coated with 200 μl conjugate and blocked with 250 μl blocking solution as above, except that the blocking solution contained 3% BSA. The plates can be stored in blocking solution at 4° C. for 1 month.

### 1.3 AngI-EIA in 384 Well MTP

The AngI (1-10)/BSA coated MTP were washed 3 times with wash buffer (PBS 1×, 0.01% Tween 20) and filled with 75 μl of primary antibody solution (anti-AngI antiserum, pre-diluted 1:10 in horse serum), diluted to a final concentration of 1:100'000 in assay buffer (PBS 1×, 1 mM EDTA, 0.1% BSA, pH 7.4). 5 μl of the renin reaction (or standards in assay buffer) (see below) were added to the primary antibody solution and the plates were incubated overnight at 4° C. After the incubation the plates were washed 3 times with wash buffer and incubated with secondary antibody [anti-rabbit IgG, linked to horseradish peroxidase (Amersham Bioscience, NA 934V), diluted 1:2'000 in wash buffer] for 2 h at rt. The plates were washed 3 times with wash buffer and then incubated for 1 h at rt with substrate solution [1.89 mM ABTS (2.2′-azino-di-(3-ethyl-benzthiazolinsulfonate)] (Roche Diagnostics, 102 946) and 2.36 mM H₂O_{2 [}30%, (Fluka, 95300] in substrate buffer (0.1M sodium acetate, 0.05M sodium dihydrogen phosphate, pH 4.2). The OD of the plate was read at 405 nm in a microplate reader (FLUOStar Optima from BMG). The production of AngI during the renin reaction was quantified by comparing the OD of the sample with the OD of a standard curve of AngI(1-10), measured in parallel.

### 2. Primary Renin Inhibition Assay: IC₅₀ in Buffer, 384 Well MTP

The renin assay was adapted from an assay described before (Fischli W. et al., Hypertension, 1991, 18:22-31) and consists of two steps: in the first step, recombinant human renin is incubated with its substrate (commercial human tetradecapeptide renin substrate) to create the product Angiotensin I (AngI). In the second step, the accumulated AngI is measured by an immunological assay (enzyme immuno assay, EIA). The detailed description of this assay is found below. The EIA is very sensitive and well suited for renin activity measurements in buffer or in plasma. Due to the low concentration of renin used in this assay (2 fmol per assay tube or 10 pM) it is possible to measure inhibitor affinities in this primary assay down to low pM concentration.

### 2.1 Methodology

Recombinant human renin (3 pg/μl) in assay buffer (PBS 1×, 1 mM EDTA, 0.1% BSA, pH 7.4), human tetradecapeptide (1-14) substrate (Bachem, M-1120) [5 μM in 10 mM HCl], hydroxyquinoline sulfate (Fluka, 55100) [30 mM in H₂O] and assay buffer were premixed at 4° C. at a ratio of 100:30:10:145. 47.5 μl per well of this premix was transferred into polypropylene plates (MTP384, Nunc). Test compounds were dissolved and diluted in 100% DMSO and 2.5 μl added to the premix, then incubated at 37° C. for 3 h. At the end of the incubation period, 5 μl of the renin reaction (or standards in assay buffer) were transferred into EIA assays (as described above) and AngI produced by renin was quantified. The percentage of renin inhibition (AngI decrease) was calculated for each concentration of compound and the concentration of renin inhibition was determined that inhibited the enzyme activity by 50% (IC₅₀). The IC₅₀-values of the compounds of the Examples are below 100 nM. Moreover, the compounds exhibit a very good bioavailability and are metabolically more stable than prior art compounds.

### Examples of Inhibition:

| Compound of | |
|---|---|
| Example No. | IC₅₀ values [nM] |
| 2 | 7.8 |
| 6 | 8.1 |
| 11 | 17 |
| 14 | 45 |
| 18 | 0.2 |
| 20 | 0.5 |
| 22 | 0.3 |

1. A compound of the formula (I) 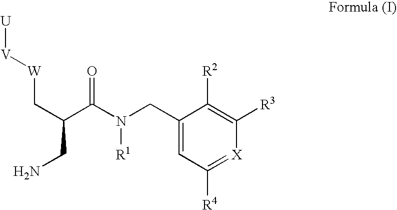
wherein
X represents CH, N, or N⁺—O⁻;
W represents a para-substituted pyridinyl or a thiazolyl;
V represents —CH₂CH₂CH₂—, —CH₂CH₂-A-, —CH₂-A-CH₂—, -A-CH₂CH₂—, —CH₂CH₂CH₂CH₂—, -A-CH₂CH₂CH₂—, —CH₂-A-CH₂CH₂—, —CH₂CH₂-A-CH₂—, —CH₂CH₂CH₂-A-, -A-CH₂CH₂—B—, —CH₂CH₂CH₂CH₂CH₂—, -A-CH₂CH₂CH₂CH₂—, —CH₂-A-CH₂CH₂CH₂—, —CH₂CH₂-A-CH₂CH₂—, —CH₂CH₂CH₂-A-CH₂—, —CH₂CH₂CH₂CH₂-A-, -A-CH₂CH₂CH₂—B—, —CH₂-A-CH₂CH₂—B—, -A-CH₂CH₂—B—CH₂—, -A-CH₂CH₂CH₂—B—CH₂—, —CH₂-A-CH₂CH₂CH₂—B—, —O—CH₂-Q-, wherein Q is bound to the group U of formula (I), or a pyrrolidinyl of the formula: 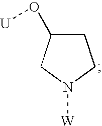
U represents unsubstituted aryl; mono-, di-, tri- or tetra-substituted aryl, wherein the substituents are independently selected from the group consisting of C₁₋₇-alkyl, —CF₃, halogen, and hydroxy-C₁₋₇-alkyl; or a five-membered heteroaryl with two heteroatoms independently selected from nitrogen, oxygen and sulphur, wherein said heteroaryl radical is optionally mono-, di- or tri-substituted, wherein the substitutents are independently selected from the group consisting of C₁₋₇-alkyl, C₁₋₇-alkoxy, —CF₃, —OCF₃, and halogen;
Q represents a five-membered heteroaryl with two or three heteroatoms independently selected from O and N;
A and B represent independently from each others —O— or —S—;
R¹ represents C₁₋₇-alkyl or cycloalkyl;
R² represents halogen or C₁₋₇-alkyl;
R³ represents hydrogen, halogen, C₁₋₇-alkyl, C₁₋₇-alkoxy, or —CF₃; and
R⁴ represents hydrogen; C₁₋₇-alkyl-O—(CH₂)₀₋₄—CH₂—; CF₃—O—(CH₂)₀₋₄—CH₂—; R′₂N—(CH₂)₀₋₄—CH₂—, wherein R′ is independently selected from the group consisting of hydrogen, C₁₋₇-alkyl (optionally substituted by one to three fluorine), cyclopropyl (optionally substituted by one to three fluorine), cyclopropyl-C₁₋₇-alkyl (optionally substituted by one to three fluorine), and —C(═O)—R″ wherein R″ is C₁₋₄-alkyl, C₁₋₄-alkoxy, —CF₃, —CH₂—CF₃, or cyclopropyl; or R⁵—C(═O)—(O)₀₋₁—(CH₂)₀₋₄—, wherein R⁵ is C₁₋₄-alkyl, C₁₋₄-alkoxy, or cyclopropyl;
and salts thereof.

2. A compound according to claim 1, wherein
X represents CH or N; and
R⁴ represents hydrogen; C₁₋₇-alkyl-O—(CH₂)₀₋₄—CH₂—; CF₃—O—(CH₂)₀₋₄—CH₂—; or R′₂N—(CH₂)₀₋₄—CH₂—, wherein R′ is independently selected from the group consisting of hydrogen, C₁₋₇-alkyl (optionally substituted by one to three fluorine), cyclopropyl (optionally substituted by one to three fluorine), cyclopropyl-C₁₋₇-alkyl (optionally substituted by one to three fluorine), and —C(═O)—R″ wherein R″ is C₁₋₄-alkyl, —CF₃, —CH₂—CF₃, or cyclopropyl;
or a salt of such a compound.

3. A compound according to claim 1, wherein X represents CH or N⁺—O⁻, or a salt of such a compound.

4. A compound according to claim 1, wherein A and B both represent —O—, or a salt of such a compound.

5. A compound according to claim 1, wherein R¹ represents cyclopropyl, or a salt of such a compound.

6. A compound according to claim 1, wherein W represents 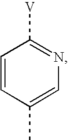
or a salt of such a compound.

7. A compound according to claim 1, wherein V represents —O—CH₂CH₂—O—, —CH₂—CH₂—O— wherein the —CH₂ part of —CH₂—CH₂—O— is bound to the group W of formula (I), —O—CH₂-Q-, or 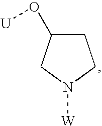
or a salt of such a compound.

8. A compound according to claim 7, wherein V represents —O—CH₂CH₂—O— or —O—CH₂-Q-, or a salt of such a compound.

9. A compound according to claim 1, wherein V—W represents: 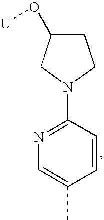
or a salt of such a compound.

10. A compound according to claim 1, wherein U represents 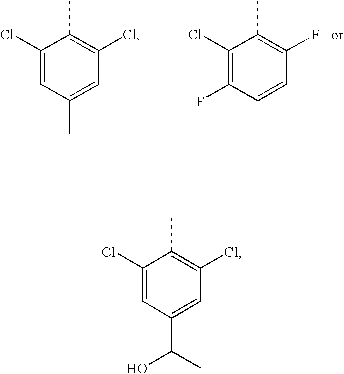
or a salt of such a compound.

11. A compound according to claim 10, wherein U represents 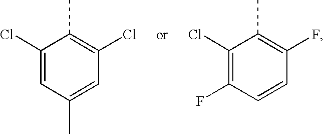
or a salt of such a compound.

12. A compound according to claim 1, wherein Q represents an isoxazolyl or an oxadiazolyl, or a salt of such a compound.

13. A compound according to claim 12, wherein Q represents an isoxazolyl, or a salt of such a compound.

14. A compound according to claim 1, wherein R² represents Cl, and R³ represents hydrogen, or a salt of such a compound.

15. A compound according to claim 1, wherein R⁴ represents CH₃—O—(CH₂)₂₋₃— or CH₃—C(═O)—NH—CH₂—CH₂—, or a salt of such a compound

16. A compound according to claim 1, wherein R⁴ represents —CH₂CH₂CH₂—O—CH₃ or —CH₂CH₂—O—CH₃, or a salt of such a compound.

17. A compound according to claim 16, wherein R⁴ represents —CH₂CH₂—O—CH₃, or a salt of such a compound.

18. A compound according to claim 1, wherein the moiety 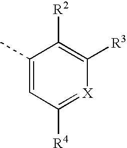
represents one of the following possibilities: 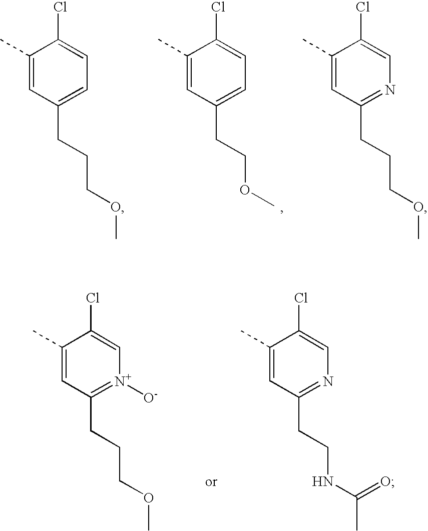
or a salt of such a compound.

19. A compound according to claim 1, wherein
X represents CH or N;
W represents a para-substituted pyridinyl or a thiazolyl;
V represents —O—CH₂CH₂—O— or a pyrrolidinyl of the formula: 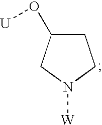
U represents di-, tri-, or tetra-substituted phenyl, wherein the substituents are independently selected from the group consisting of C₁₋₇-alkyl, halogen and hydroxy-C₁₋₇-alkyl;
R¹ represents cyclopropyl;
R² represents halogen or C₁₋₇-alkyl;
R³ represents hydrogen, halogen, or C₁₋₇-alkyl; and
R⁴ represents hydrogen or C₁₋₇-alkyl-O—(CH₂)₀₄—CH₂—;
or a salt of such a compound.

20. A compound according to claim 1 selected from the group consisting of:
(R)-3-amino-N-cyclopropyl-2-{2-[2-(2,6-dichloro-4-methyl-phenoxy)-ethoxy]-thiazol-5-ylmethyl}-N-(2,3-dimethyl-benzyl)-propionamide,
(R)-3-amino-2-{2-[2-(2-chloro-3,6-difluoro-phenoxy)-ethoxy]-thiazol-5-ylmethyl}-N-cyclopropyl-N-(2,3-dimethyl-benzyl)-propionamide,
(R)-3-amino-N-cyclopropyl-2-{2-[2-(2,6-dichloro-phenoxy)-ethoxy]-thiazol-5-ylmethyl}-N-(2,3-dimethyl-benzyl)-propionamide,
(R)-3-amino-N-cyclopropyl-2-{2-[2-(2,6-dichloro-3,4-dimethyl-phenoxy)-ethoxy]-thiazol-5-ylmethyl}-N-(2,3-dimethyl-benzyl)-propionamide,
(R)-3-amino-2-{2-[2-(2-chloro-6-fluoro-3-methyl-phenoxy)-ethoxy]-thiazol-5-ylmethyl}-N-cyclopropyl-N-(2,3-dimethyl-benzyl)-propionamide,
(R)-3-amino-N-cyclopropyl-2-(2-{2-[2,6-dichloro-4-(1-hydroxy-ethyl)-phenoxy]-ethoxy}-thiazol-5-ylmethyl)-N-(2,3-dimethyl-benzyl)-propionamide,
(R)-3-amino-2-{2-[2-(3-chloro-2,6-difluoro-phenoxy)-ethoxy]-thiazol-5-ylmethyl}-N-cyclopropyl-N-(2,3-dimethyl-benzyl)-propionamide,
(R)-3-amino-N-cyclopropyl-2-{2-[2-(2,6-dichloro-4-fluoro-phenoxy)-ethoxy]-thiazol-5-ylmethyl}-N-(2,3-dimethyl-benzyl)-propionamide,
(R)-2-aminomethyl-N-cyclopropyl-N-(2,3-dichloro-benzyl)-3-{2-[(R)-3-(2,6-dichloro-4-methyl-phenoxy)-pyrrolidin-1-yl]-thiazol-5-yl}-propionamide,
(R)-2-aminomethyl-N-cyclopropyl-N-(2,3-dichloro-benzyl)-3-{2-[(R)-3-(2,6-dichloro-phenoxy)-pyrrolidin-1-yl]-thiazol-5-yl}-propionamide,
(R)-2-aminomethyl-N-cyclopropyl-N-(2,3-dichloro-benzyl)-3-{2-[(R)-3-(2,6-dichloro-3,4-dimethyl-phenoxy)-pyrrolidin-1-yl]-thiazol-5-yl}-propionamide,
(R)-2-aminomethyl-3-{2-[(R)-3-(2-chloro-6-fluoro-3-methyl-phenoxy)-pyrrolidin-1-yl]-thiazol-5-yl}-N-cyclopropyl-N-(2,3-dichloro-benzyl)-propionamide,
(R)-2-aminomethyl-N-cyclopropyl-N-(2,3-dichloro-benzyl)-3-(2-{(R)-3-[(R)-2,6-dichloro-4-(1-hydroxy-ethyl)-phenoxy]-pyrrolidin-1-yl}-thiazol-5-yl)-propionamide,
(R)-2-aminomethyl-N-cyclopropyl-N-(2,3-dichloro-benzyl)-3-(2-{(R)-3-[(S)-2,6-dichloro-4-(1-hydroxy-ethyl)-phenoxy]-pyrrolidin-1-yl}-thiazol-5-yl)-propionamide,
(R)-2-aminomethyl-3-{2-[(R)-3-(3-chloro-2,6-difluoro-phenoxy)-pyrrolidin-1-yl]-thiazol-5-yl}-N-cyclopropyl-N-(2,3-dichloro-benzyl)-propionamide,
(R)-2-aminomethyl-N-cyclopropyl-N-(2,3-dichloro-benzyl)-3-{2-[(R)-3-(2,6-dichloro-4-fluoro-phenoxy)-pyrrolidin-1-yl]-thiazol-5-yl}-propionamide,
(R)-2-aminomethyl-N-[2-chloro-5-(3-methoxy-propyl)-benzyl]-N-cyclopropyl-3-{6-[(R)-3-(2,6-dichloro-4-methyl-phenoxy)-pyrrolidin-1-yl]-pyridin-3-yl}-propionamide, and
(R)-2-aminomethyl-N-[5-chloro-2-(3-methoxy-propyl)-pyridin-4-ylmethyl]-N-cyclopropyl-3-{6-[(R)-3-(2,6-dichloro-4-methyl-phenoxy)-pyrrolidin-1-yl]-pyridin-3-yl}-propionamide,
and salts of these compounds.

21. A compound according to claim 1 selected from the group consisting of:
(R)-2-aminomethyl-N-[2-chloro-5-(2-methoxy-ethyl)-benzyl]-N-cyclopropyl-3-{6-[2-(2,6-dichloro-4-methyl-phenoxy)-ethoxy]-pyridin-3-yl}-propionamide,
(R)-2-aminomethyl-N-[2-chloro-5-(3-methoxy-propyl)-benzyl]-N-cyclopropyl-3-{6-[2-(2,6-dichloro-4-methyl-phenoxy)-ethoxy]-pyridin-3-yl}-propionamide,
(R)-2-aminomethyl-N-[2-chloro-5-(2-methoxy-ethyl)-benzyl]-N-cyclopropyl-3-{6-[(R)-3-(2,6-dichloro-4-methyl-phenoxy)-pyrrolidin-1-yl]-pyridin-3-yl}-propionamide,
(R)-2-aminomethyl-N-[5-chloro-2-(3-methoxy-propyl)-pyridin-4-ylmethyl]-N-cyclopropyl-3-{6-[2-(2,6-dichloro-4-methyl-phenoxy)-ethoxy]-pyridin-3-yl}-propionamide, and
(R)-2-aminomethyl-N-[2-chloro-5-(2-methoxy-ethyl)-benzyl]-N-cyclopropyl-3-{6-[(S)-3-(2,6-dichloro-4-methyl-phenoxy)-pyrrolidin-1-yl]-pyridin-3-yl}-propionamide,
and salts of these compounds.

22. A pharmaceutical composition comprising a compound according to claim 1, or a pharmaceutically acceptable salt thereof, and a pharmaceutically acceptable carrier material.

23. (canceled)

24. A method for treating a disease selected from hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intraocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases related to the renin-angiotensin system, comprising administering to a patient in need thereof the composition of claim 1.
